(19) United States
(12) Patent Application Publication
(10) Pub. No.: US 2016/0221989 A1
(43) Pub. Date: Aug. 4, 2016

(54) N-Acylpyrrolidine Ether Tropomyosin-Related Kinase Inhibitors

(71) Applicant: Pfizer Inc., New York, NY (US)

(72) Inventors: Sharanjeet Kaur Bagal, Great Abington (GB); Kiyoyuki Omoto, Great Abington (GB); Sarah Elizabeth Skerratt, Great Abington (GB); Jingrong Jean Cui, San Diego, CA (US); Samantha Elizabeth Greasley, San Diego, CA (US); Indrawan James McAlpine, San Diego, CA (US); Asako Nagata, San Diego, CA (US); Sacha Ninkovic, La Jolla, CA (US); Michelle Bich Tran-Dubé, San Diego, CA (US)

(73) Assignee: Pfizer Inc., New York, NY (US)

(21) Appl. No.: 14/817,416

(22) Filed: Aug. 4, 2015

Related U.S. Application Data
(60) Provisional application No. 62/033,158, filed on Aug. 5, 2014.

(51) Int. Cl. C07D 401/12; A61K 31/4439

(52) U.S. Cl. CPC C07D 401/12; A61K 31/4439

(57) ABSTRACT

The present invention relates to compounds of Formula I

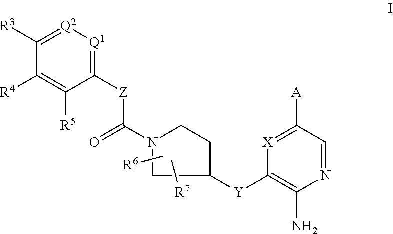

described herein and their pharmaceutically acceptable salts, and their use in medicine, in particular as Trk antagonists.

This application claims the benefit under 35 U.S.C. §119(e) to U.S. Provisional Patent Application No. 62/033,158, filed on Aug. 5, 2014, the disclosure of which is hereby incorporated by reference in its entirety.

### FIELD OF THE INVENTION

The invention described herein relates to certain piperidine compounds and the pharmaceutically acceptable salts of such compounds. The invention also relates to the processes for the preparation of the compounds, compositions containing the compounds, and the uses of such compounds and salts in treating diseases or conditions associated with tropomyosin-related kinase (Trk), activity. More specifically the invention relates to the compounds and their salts useful as inhibitors of Trk.

### BACKGROUND

Tropomyosin-related kinases (Trks) are a family of receptor tyrosine kinases activated by neurotrophins. Trks play important roles in pain sensation as well as tumour cell growth and survival signaling. Thus, inhibitors of Trk receptor kinases might provide targeted treatments for conditions such as pain and cancer. Recent developments in this field have been reviewed by Wang et al in Expert Opin. Ther. Patents (2009) 19(3): 305-319, and McCarthy et al in Expert Opin. Ther. Patents (2014) 24(7): 731-744. An extract from Wang is reproduced below.

### “1.1 Trk Receptors

As one of the largest family of proteins encoded by the human genome, protein kinases are the central regulators of signal transduction as well as control of various complex cell processes. Receptor tyrosine kinases (RTKs) are a subfamily of protein kinases (up to 100 members) bound to the cell membrane that specifically act on the tyrosine residues of proteins. One small group within this subfamily is the Trk kinases, with three highly homologous isoforms: TrkA, TrkB, and TrkC. All three isoforms are activated by high affinity growth factors named neurotrophins (NT): i) nerve growth factor (NGF), which activates TrkA; ii) brain-derived neurotrophic factor (BDNF) and NT-4/5, which activate TrkB; and iii) NT-3, which activates TrkC. The binding of neurotrophins to the extracellular domain of Trks causes the Trk kinase to autophosphorylate at several intracellular tyrosine sites and triggers downstream signal transduction pathways. Trks and neurotrophins are well known for their effects on neuronal growth and survival.

### 1.2 Trks and Cancer

Originally isolated from neuronal tissues, Trks were thought to mainly affect the maintenance and survival of neuronal cells. However, in the past 20 years, increasing evidence has suggested that Trks play key roles in malignant transformation, chemotaxis, metastasis, and survival signaling in human tumors. The association between Trks and cancer focused on prostate cancer in earlier years and the topic has been reviewed. For example, it was reported that malignant prostate epithelial cells secrete a series of neurotrophins and at least one Trks. In pancreatic cancer, it was proposed that paracrine and/or autocrine neurotrophin-Trk interactions may influence the invasive behavior of the cancer. TrkB was also reported to be overexpressed in metastatic human pancreatic cancer cells. Recently, there have been a number of new findings in other cancer settings. For example, a translocation leads to expression of a fusion protein derived from the N-terminus of the ETV6 transcription factor and the C-terminal kinase domain of TrkC. The resulting ETV6-TrkC fusions are oncogenic in vitro and appear causative in secretory breast carcinoma and some acute myelogenous leukemias (AML). Constitutively active TrkA fusions occurred in a subset of papillary thyroid cancers and colon carcinomas. In neuroblastoma, TrkB expression was reported to be a strong predictor of aggressive tumor growth and poor prognosis, and TrkB overexpression was also associated with increased resistance to chemotherapy in neuroblastoma tumor cells in vitro. One report showed that a novel splice variant of TrkA called TrkAIII signaled in the absence of neurotrophins through the inositol phosphate-AKT pathway in a subset of neuroblastoma. Also, mutational analysis of the tyrosine kinome revealed that Trk mutations occurred in colorectal and lung cancers. In summary, Trks have been linked to a variety of human cancers, and discovering a Trk inhibitor and testing it clinically might provide further insight to the biological and medical hypothesis of treating cancer with targeted therapies.

### 1.3 Trks and Pain

Besides the newly developed association with cancer, Trks are also being recognized as an important mediator of pain sensation. Congenital insensitivity to pain with anhidrosis (CIPA) is a disorder of the peripheral nerves (and normally innervated sweat glands) that prevents the patient from either being able to adequately perceive painful stimuli or to sweat. TrkA defects have been shown to cause CIPA in various ethnic groups.

Currently, non-steroidal anti-inflammatory drugs (NSAIDs) and opiates have low efficacy and/or side effects (e.g., gastrointestinal/renal and psychotropic side effects, respectively) against neuropathic pain and therefore development of novel pain treatments is highly desired. It has been recognized that NGF levels are elevated in response to chronic pain, injury and inflammation and the administration of exogenous NGF increases pain hypersensitivity. In addition, inhibition of NGF function with either anti-NGF antibodies or non-selective small molecule Trk inhibitors has been shown to have effects on pain in animal models. It appears that a selective Trk inhibitor (inhibiting at least NGF's target, the TrkA receptor) might provide clinical benefit for the treatment of pain. Excellent earlier reviews have covered targeting NGF/BDNF for the treatment of pain so this review will only focus on small molecule Trk kinase inhibitors claimed against cancer and pain. However, it is notable that the NGF antibody tanezumab was very recently reported to show good efficacy in a Phase II trial against osteoarthritic knee pain.”

Further trk-mediated conditions which have been investigated and show promise for treatment with a trk inhibitor include atopic dermatitis, psoriasis, eczema and prurigo nodularis, acute and chronic itch, pruritis, atopic dermatitis, inflammation, cancer, restenosis, atherosclerosis, psoriasis, thrombosis, pruritis, lower urinary tract disorder, inflammatory lung diseases such as asthma, allergic rhinitis, lung cancer, psoriatic arthritis, rheumatoid arthritis, inflammatory bowel diseases such as ulcerative colitis, Crohn's disease, fibrosis, neurodegenerative disease, diseases disorders and conditions related to dysmyelination or demyelination, certain infectious diseases such as Trypanosoma cruzi infection (Chagas disease), cancer related pain, chronic pain, neuroblastoma, ovarian cancer, colorectal cancer, melanoma, head and neck cancer, gastric carcimoma, lung carcinoma, breast cancer, glioblastoma, medulloblastoma, secratory breast cancer, salivary gland cancer, papillary thyroid carcinoma, adult myeloid leukaemia, tumour growth and metastasis, interstitial cystitis and Alzheimer's disease (McCarthy et al in Expert Opin. Ther. Patents (2014) 24(7): 731-744; C. Potenzieri and B. J. Undem, Clinical & Experimental Allergy, 2012 (42) 8-19; Yamaguchi J, Aihara M, Kobayashi Y, Kambara T, Ikezawa Z, J Dermatol Sci. 2009; 53:48-54; Dou Y C, Hagstromer L, Emtestam L, Johansson O., Arch Dermatol Res. 2006; 298:31-37; Johansson O, Liang Y, Emtestam L., Arch Dermatol Res. 2002; 293:614-619; Grewe M, Vogelsang K, Ruzicka T, Stege H, Krutmann J., J Invest Dermatol. 2000; 114:1108-1112; Urashima R, Mihara M. Virchows Arch. 1998; 432:363-370; Kinkelin I, Motzing S, Koltenzenburg M, Brocker E B., Cell Tissue Res. 2000; 302:31-37; Tong Liu & Ru-Rong Ji, Pflugers Arch—Eur J Physiol, DOI 10.1007/s00424-013-1284-2, published online 1 May 2013.); International Patent Application publication numbers WO2012/158413, WO2013/088256, WO2013/088257 and WO2013/161919, (Brodeur, G. M., Nat. Rev. Cancer 2003, 3, 203-216), (Davidson. B., et al., Clin. Cancer Res. 2003, 9, 2248-2259), (Bardelli, A., Science 2003, 300, 949), (Truzzi, F., et al., Dermato-Endocrinology 2008, 3 (I), pp. 32-36), Yilmaz, T., et al., Cancer Biology and Therapy 2010, 10 (6), pp. 644-653), (Du, J. et al., World Journal of Gastroenterology 2003, 9 (7), pp. 1431-1434), (Ricci A., et al., American Journal of Respiratory Cell and Molecular Biology 25 (4), pp. 439-446), (Jin, W., et al., Carcinogenesis 2010, 31 (11), pp. 1939-1947), (Wadhwa, S., et al., Journal of Biosciences 2003, 28 (2), pp. 181-188), (Gruber-Olipitz, M., et al., Journal of Proteome Research 2008, 7 (5), pp. 1932-1944), (Euthus, D. M. et al., Cancer Cell 2002, 2 (5), pp. 347-348), (Li, Y.-G., et al., Chinese Journal of Cancer Prevention and Treatment 2009, 16 (6), pp. 428-430), (Greco, A., et al., Molecular and Cellular Endocrinology 2010, 321 (I), pp. 44-49), (Eguchi, M., et al., Blood 1999, 93 (4), pp. 1355-1363), (Nakagawara, A. (2001) Cancer Letters 169:107-114; Meyer, J. et al. (2007) Leukemia, 1-10; Pierottia, M. A. and Greco A., (2006) Cancer Letters 232:90-98; Eric Adriaenssens, E., et al. Cancer Res (2008) 68:(2) 346-351), (Freund-Michel, V; Frossard, N., Pharmacology ck Therapeutics (2008) 117(1), 52-76), (Hu Vivian Y; et. al. The Journal of Urology (2005), 173(3), 1016-21), (Di Mola, F. F, et. al. Gut (2000) 46(5), 670-678) (Dou, Y.-C., et. al. Archives of Dermatological Research (2006) 298(1), 31-37), (Raychaudhuri, S. P., et al., J. Investigative Dermatology (2004) 122(3), 812-819) and (de Melo-Jorge, M. et al., Cell Host ck Microbe (2007) 1(4), 251-261).

Thus Trk inhibitors have a wide variety of potential medical uses. There is a need to provide new Trk inhibitors that are good drug candidates. In particular, compounds should preferably bind potently to the Trk receptors in a selective manner compared to other receptors, whilst showing little affinity for other receptors, including other kinase and/or GPC receptors, and show functional activity as Trk receptor antagonists. They should be non-toxic and demonstrate few side-effects. Furthermore, the ideal drug candidate will exist in a physical form that is stable, non-hygroscopic and easily formulated. They should preferably be e.g. well absorbed from the gastrointestinal tract, and/or be injectable directly into the bloodstream, muscle, or subcutaneously, and/or be metabolically stable and possess favourable pharmacokinetic properties.

International Patent Application publication number WO2009/012283 refers to various fluorophenyl compounds as Trk inhibitors; International Patent Application publication numbers WO2009/152087, WO2008/080015 and WO2008/08001 and WO2009/152083 refer to various fused pyrroles as kinase modulators; International Patent Application publication numbers WO2009/143024 and WO2009/143018 refer to various pyrrolo[2,3-d]pyrimidines substituted as Trk inhibitors; International Patent Application publication numbers WO2004/056830 and WO2005/116035 describe various 4-amino-pyrrolo[2,3-d]pyrimidines as Trk inhibitors. International Patent Application publication number WO2011/133637 describes various pyrrolo[2,3-d]pyrimidines and pyrrolo[2,3-b]pyridines as inhibitors of various kinases. International Patent Application publication number WO2005/099709 describes bicyclic heterocycles as serine protease inhibitors. International Patent Application publication number WO2007/047207 describes bicyclic heterocycles as FLAP modulators.

International Patent Application publication numbers WO2012/137089, WO2014/053967, WO2014/053968 and WO2014/053965 describe various heterocyclic compounds as Trk inhibitors.

Among the aims of this invention are to provide orally-active, efficacious, compounds and salts which can be used as active drug substances, particularly Trk antagonists, i.e. that block the intracellular kinase activity of the Trk, e.g. TrkA (NGF) receptor. Other desirable features include good HLM/hepatocyte stability, oral bioavailability, metabolic stability, absorption, selectivity over other types of kinase, dofetilide selectivity. Preferable compounds and salts will show a lack of CYP inhibition/induction, and be CNS-sparing.

### SUMMARY

The present invention provides compounds of Formula I:

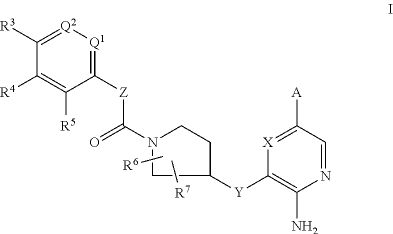

### Wherein

Q¹ is N or CR¹,
Q² is N or CR²,
R¹, R², R⁴ and R⁵ are each independently H, halogen, CN, OH, NH₂, C₁₋₃ alkyl optionally substituted by one or more F, C₃₋₇ cycloalkyloxy optionally substituted by one or more F, or C₁₋₃ alkoxy optionally substituted by one or more F,
R³ is H, halogen, CN, C₁₋₄ alkyl optionally substituted by one or more F, C₁₋₄ alkoxy optionally substituted by one or more F, C₃₋₇ cycloalkyloxy optionally substituted by one or more F, or C₁₋₄ alkylthio optionally substituted by one or more F,
With the proviso that at least 2 of R¹, R², R³, R⁴ and R⁵ are H,
Y is O, CH₂O or OCH₂
R⁶ and R⁷ can be attached at any point on the ring and are independently H, F, CN, OH,
NH₂, C₁₋₃ alkyl optionally substituted by one or more F, or C₁₋₃ alkoxy optionally substituted by one or more F,
or R⁶ and R⁷ can be taken together, with the atoms to which they are attached, to form a 3- to 7-membered cycloalkane ring,

### X is CR¹⁰¹ or N,

R¹⁰¹ is H or C₁₋₃ alkyl,
Z is CH₂, CH(CH₃), NH or O,
A is C(O)NR¹⁰³R¹⁰⁴,
R¹⁰³ and R¹⁰⁴ are each independently selected from H, (C₁₋₆ alkyl optionally substituted by OH, C₁₋₆ alkoxy, CN or by one or more F), and (C₃₋₇ cycloalkyl optionally substituted by OH, C₁₋₆ alkoxy or by one or more F),
and pharmaceutically acceptable salts thereof.

The invention also comprises pharmaceutical compositions comprising a therapeutically effective amount of a compound of formula I as defined herein, or a pharmaceutically acceptable salt thereof, and a pharmaceutically acceptable carrier.

The invention is also directed to a method of treating a disease or condition indicated for treatment with a Trk antagonist, in a subject, by administering to a subject in need thereof a therapeutically effective amount of one or more of the compounds herein, or a pharmaceutically acceptable salt thereof.

Other aspects of the invention will be apparent from the remaining description and claims.

Preferably, the compounds of the present invention are potent antagonists at Trk receptors, and have a suitable PK profile to enable once daily, twice daily, or thrice daily dosing.

The compounds of the present invention are potentially useful in the treatment of a range of disorders where a Trk antagonist is indicated, particularly pain indications. Depending on the disease and condition of the patient, the term “treatment” as used herein may include one or more of curative, palliative and prophylactic treatment.

According to the invention a compound of the present invention may be useful to treat any physiological pain such as inflammatory pain, nociceptive pain, neuropathic pain, acute pain, chronic pain, musculo-skeletal pain, on-going pain, central pain, heart and vascular pain, head pain, orofacial pain. Other pain conditions which may be treated include intense acute pain and chronic pain conditions which may involve the same pain pathways driven by pathophysiological processes and as such cease to provide a protective mechanism and instead contribute to debilitating symptoms associated with a wide range of disease states.

Pain is a feature of many trauma and disease states. When a substantial injury, via disease or trauma, to body tissue occurs the characteristics of nociceptor activation are altered, this leads to hypersensitivity at the site of damage and in nearby normal tissue. In acute pain the sensitivity returns to normal once the injury has healed. However, in many chronic pain states, the hypersensitivity far outlasts the healing process and is normally due to nervous system injury due to maladaptation of the afferent fibres (Woolf & Salter 2000 Science 288: 1765-1768). Clinical pain is present when discomfort and abnormal sensitivity feature among the patient's symptoms. There are a number of typical pain subtypes: 1) spontaneous pain which may be dull, burning, or stabbing; 2) pain responses to noxious stimuli are exaggerated (hyperalgesia); 3) pain is produced by normally innocuous stimuli (allodynia) (Meyer et al., 1994 Textbook of Pain 13-44). Pain can be divided into a number of different areas because of differing pathophysiology, these include nociceptive, inflammatory, neuropathic pain among others. It should be noted that some types of pain have multiple aetiologies and thus can be classified in more than one area, e.g. Back pain, Cancer pain have both nociceptive and neuropathic components.

Disorders for which a trk inhibitor may be indicated include pain. Pain may be either acute or chronic and additionally may be of central and/or peripheral origin. Pain may be of a neuropathic and/or nociceptive and/or inflammatory nature, such as pain affecting either the somatic or visceral systems, as well as dysfunctional pain affecting multiple systems.

Physiological pain is an important protective mechanism designed to warn of danger from potentially injurious stimuli from the external environment. The system operates through a specific set of primary sensory neurones and is activated by noxious stimuli via peripheral transducing mechanisms (see Meyer et al., 2006, Wall and Melzack's Textbook of Pain (5^{th} Ed), Chapter 1). These sensory fibres are known as nociceptors, and are characteristically small diameter axons with slow conduction velocities, of which there are two main types, A-delta fibres (myelinated) and C fibres (non-myelinated). Nociceptors encode the intensity, duration and quality of noxious stimulus and by virtue of their topographically organised projection to the spinal cord, the location of the stimulus. The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually, although not always, associated with a specific cause such as a defined injury, is often sharp and severe and can result from numerous origins such as surgery, dental work, a strain or a sprain. Acute pain does not generally result in any persistent psychological response. When a substantial injury occurs to body tissue, via disease or trauma, the characteristics of nociceptor activation may be altered such that there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury or alteration which can be associated with maladaptation and aberrant activity (Woolf & Salter, 2000, Science, 288, 1765-1768). As such, chronic pain is long-term pain, typically persisting for more than three months and leading to significant psychological and emotional problems. Common examples of chronic pain are neuropathic pain (e.g. painful diabetic neuropathy or postherpetic neuralgia), carpal tunnel syndrome, back pain, headache, cancer pain, arthritic pain and chronic post-surgical pain, but may include any chronic painful condition affecting any system, such as those described by the International Association for the Study of Pain (Classification of Chronic Pain, a publication freely available for download at http://www.iasp-pain.org).

The clinical manifestation of pain is present when discomfort and abnormal sensitivity feature among the patient's symptoms. Patients tend to be quite heterogeneous and may present with various pain symptoms. Such symptoms can include: 1) spontaneous pain which may be dull, burning, or stabbing; 2) exaggerated pain responses to noxious stimuli (hyperalgesia); and 3) pain produced by normally innocuous stimuli (allodynia) (Meyer et al., 2006, Wall and Melzack's Textbook of Pain (5^{th} Ed), Chapter 1). Although patients suffering from various forms of acute and chronic pain may have similar symptoms, the underlying mechanisms may be different and may, therefore, require different treatment strategies. Apart from acute or chronic, pain can also be broadly categorized into: nociceptive pain, affecting either the somatic or visceral systems, which can be inflammatory in nature (associated with tissue damage and the infiltration of immune cells); or neuropathic pain.

Nociceptive pain can be defined as the process by which intense thermal, mechanical, or chemical stimuli are detected by a subpopulation of peripheral nerve fibers, called nociceptors, and can be induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 2006, Wall and Melzack's Textbook of Pain (5^{th} Ed), Chapter 1). Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from strains/sprains, burns, myocardial infarction and acute pancreatitis, post-operative pain (pain following any type of surgical procedure), posttraumatic pain, pain associated with gout, cancer pain and back pain. Cancer pain may be chronic pain such as tumour related pain (e.g. bone pain, headache, facial pain or visceral pain) or pain associated with cancer therapy (e.g. in response to chemotherapy, immunotherapy, hormonal therapy or radiotherapy). Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Nociceptive pain can also be related to inflammatory states. The inflammatory process is a complex series of biochemical and cellular events, activated in response to tissue injury or the presence of foreign substances, which results in swelling and pain (McMahon et al., 2006, Wall and Melzack's Textbook of Pain (5^{th} Ed), Chapter 3). A common inflammatory condition assoiciated with pain is arthritis. It has been estimated that almost 27 million Americans have symptomatic osteoarthritis (OA) or degenerative joint disease (Lawrence et al., 2008, Arthritis Rheum, 58, 15-35); most patients with osteoarthritis seek medical attention because of the associated pain. Arthritis has a significant impact on psychosocial and physical function and is known to be the leading cause of disability in later life. Rheumatoid arthritis is an immune-mediated, chronic, inflammatory polyarthritis disease, mainly affecting peripheral synovial joints. It is one of the commonest chronic inflammatory conditions in developed countries and is a major cause of pain.

In regard to nociceptive pain of visceral origin, visceral pain results from the activation of nociceptors of the thoracic, pelvic, or abdominal organs (Bielefeldt and Gebhart, 2006, Wall and Melzack's Textbook of Pain (5^{th} Ed), Chapter 48). This includes the reproductive organs, spleen, liver, gastrointestinal and urinary tracts, airway structures, cardiovascular system and other organs contained within the abdominal cavity. As such visceral pain refers to pain associated with conditions of such organs, such as painful bladder syndrome, interstitial cystitis, prostatitis, ulcerative colitis, Crohn's disease, renal colic, irritable bowl syndrome, endometriosis and dysmenorrheal (Classification of Chronic Pain, available at http://www.iasp-pain.org). Currently the potential for a neuropathic contribution (either through central changes or nerve injury/damage) to visceral pain states is poorly understood but may play a role in certain conditions (Aziz et al., 2009, Dig Dis 27, Suppl 1, 31-41)

Neuropathic pain is currently defined as pain arising as a direct consequence of a lesion or disease affecting the somatosensory system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Dworkin, 2009, Am J Med, 122, S1-S2; Geber et al., 2009, Am J Med, 122, S3-S12; Haanpaa et al., 2009, Am J Med, 122, S13-S21). The symptoms of neuropathic pain are difficult to treat, as they are often heterogeneous even between patients with the same disease (Dworkin, 2009, Am J Med, 122, S1-S2; Geber et al., 2009, Am J Med, 122, S3-S12; Haanpaa et al., 2009, Am J Med, 122, S13-S21). They include spontaneous pain, which can be continuous, and paroxysmal or abnormal evoked pain, such as hyperalgesia (increased sensitivity to a noxious stimulus) and allodynia (sensitivity to a normally innocuous stimulus).

It should be noted that some types of pain have multiple aetiologies and thus can be classified in more than one area, e.g. back pain, cancer pain and even migaine headaches may include both nociceptive and neuropathic components.

Similarly other types of chronic pain, perhaps less well understood, are not easily defined by the simplistic definitions of nociceptive or neuropathic. Such conditions include in particular fibromyalgia and chronic regional pain syndrome, which are often described as dysfunctional pain states e.g. fibromyalgia or complex regional pain syndrome (Woolf, 2010, J Clin Invest, 120, 3742-3744), but which are included in classifications of chronic pain states (Classification of Chronic Pain, available at http://www.iasp-pain.org).

### DETAILED DESCRIPTION

Embodiment 1 of the invention is a compound of Formula I:

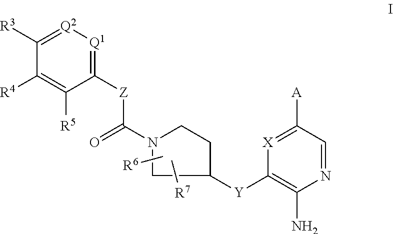

### Wherein

Q¹ is N or CR¹,
Q² is N or CR²,
R¹, R², R⁴ and R⁵ are each independently H, halogen, CN, OH, NH₂, C₁₋₃ alkyl optionally substituted by one or more F, C₃₋₇ cycloalkyloxy optionally substituted by one or more F, or C₁₋₃ alkoxy optionally substituted by one or more F,
R³ is H, halogen, CN, C₁₋₄ alkyl optionally substituted by one or more F, C₁₋₄ alkoxy optionally substituted by one or more F, C₃₋₇ cycloalkyloxy optionally substituted by one or more F, or C₁₋₄ alkylthio optionally substituted by one or more F,
With the proviso that at least 2 of R¹, R², R³, R⁴ and R⁵ are H,
Y is O, CH₂O or OCH₂
R⁶ and R⁷ can be attached at any point on the ring and are independently H, F, CN, OH, NH₂, C₁₋₃ alkyl optionally substituted by one or more F, or C₁₋₃ alkoxy optionally substituted by one or more F,
or R⁶ and R⁷ can be taken together, with the atoms to which they are attached, to form a 3- to 7-membered cycloalkane ring,

### X is CR¹⁰¹ or N,

R¹⁰¹ is H or C₁₋₃ alkyl,
Z is CH₂, CH(CH₃), NH or O,
A is C(O)NR¹⁰³R¹⁰⁴,
R¹⁰³ and R¹⁰⁴ are each independently selected from H, (C₁₋₆ alkyl optionally substituted by OH, C₁₋₆ alkoxy, CN or by one or more F), and (C₃₋₇ cycloalkyl optionally substituted by OH, C₁₋₆ alkoxy or by one or more F), and pharmaceutically acceptable salts thereof.

### Embodiment 2

A compound or salt according to embodiment 1 wherein Q¹ is CH

### Embodiment 3

A compound or salt according to embodiment 1 or 2 wherein Q² is CH

### Embodiment 4

A compound or salt according to embodiment 1, 2 or 3 wherein R⁴ is H

### Embodiment 5

A compound or salt according to embodiment 1, 2, 3 or 4 wherein R⁵ is H.

### Embodiment 6

A compound or salt according to embodiment 1, 2, 3, 4 or 5 wherein R³ is halogen, C₁₋₄ alkyl optionally substituted by one or more F, C₁₋₄ alkoxy optionally substituted by one or more F, or C₃₋₇ cycloalkyloxy optionally substituted by one or more F, or C₁₋₄ alkylthio optionally substituted by one or more F.

### Embodiment 7

A compound or salt according to embodiment 6 wherein R³ is C₁₋₄ alkoxy optionally substituted by one or more F.

### Embodiment 8

A compound or salt according to embodiment 7 wherein R³ is OCF₃

### Embodiment 9

A compound or salt according to any one of embodiments 1, 2, 3, 4, 5, 7 or 8 wherein R⁶ and R⁷ can be attached at any point on the ring and are independently H, F, methyl optionally substituted by one or more F, ethyl optionally substituted by one or more F, or methoxy optionally substituted by one or more F, or R⁶ and R⁷ can be taken together, with the atoms to which they are attached, to form a cyclopropyl ring.

### Embodiment 10

A compound or salt according to embodiment 9 wherein R⁶ is H, F or methyl and R⁷ is F, methyl or methoxy, or R⁶ and R⁷ together are cyclopropyl.

### Embodiment 11

A compound or salt according to any one of embodiments 1, 2, 3, 4, 5, 6, 7, 8, 9 or 10 wherein X is CR¹⁰¹.

### Embodiment 12

A compound or salt according to embodiment 11 wherein X is CH Embodiment 13. A compound or salt according to any one of embodiments 1, 2, 3, 4, 5, 6, 7, 8, 9, 10, 11 or 12 wherein Y is O.

### Embodiment 14

A compound or salt according to any one of embodiments 1, 2, 3, 4, 5, 6, 7, 8, 9, 10, 11, 12 or 13 wherein R¹⁰³ and R¹⁰⁴ are each independently selected from H and C₁₋₆ alkyl optionally substituted by OH or CN.

### Embodiment 15

A compound or salt according to embodiment 14 wherein R¹⁰³ is H, methyl or ethyl.

### Embodiment 16

A compound or salt according to embodiment 14 or 15 wherein R¹⁰⁴ is selected from H, methyl, ethyl, 2-hydroxyethyl, 2,2-dimethyl-2-hydroxyethyl or cyanomethyl.

### Embodiment 17

A compound according to embodiment 1 of formula IA:

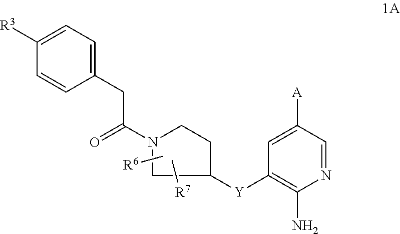

Or a pharmaceutically acceptable salt thereof.

### Embodiment 18

A compound or salt according to any one of embodiments 1, 2, 3, 4, 5, 6, 7, 8, 9, 10, 11, 12, 13, 14, 15, 16 or 17 which is of formula IB

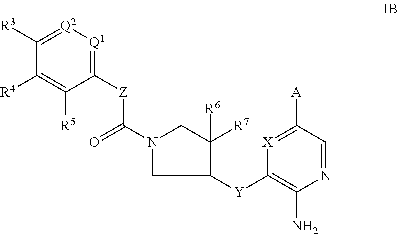

Or a pharmaceutically acceptable salt thereof.

### Embodiment 19

A compound selected from any of the Examples below, or a pharmaceutically acceptable salt thereof.

### Embodiment 20

A pharmaceutical composition comprising a compound of the formula (I) or a pharmaceutically acceptable salt thereof, as defined in any one of the preceding embodiments 1 to 19, and a pharmaceutically acceptable carrier.

### Embodiment 21

A compound of the formula (I) or a pharmaceutically acceptable salt thereof, as defined in any one of embodiments 1 to 19, for use as a medicament.

### Embodiment 22

A compound of formula (I) or a pharmaceutically acceptable salt thereof, as defined in any one of embodiments 1 to 19 for use in the treatment of a disease for which a Trk receptor antagonist is indicated.

### Embodiment 23

A compound of formula (I) or a pharmaceutically acceptable salt thereof, as defined in any one of embodiments 1 to 19 for use in the treatment of pain or cancer.

### Embodiment 24

The use of a compound of the formula (I) or a pharmaceutically acceptable salt or composition thereof, as defined in any one of embodiments 1 to 19, for the manufacture of a medicament to treat a disease for which a Trk receptor antagonist is indicated

### Embodiment 25

The use of a compound of the formula (I) or a pharmaceutically acceptable salt or composition thereof, as defined in any one of embodiments 1 to 19, for the manufacture of a medicament to treat pain or cancer.

### Embodiment 26

A method of treatment of a mammal, to treat a disease for which a Trk receptor antagonist is indicated, comprising treating said mammal with an effective amount of a compound of the formula (I) or a pharmaceutically acceptable salt thereof, as defined in any one of embodiments 1 to 19.

### Embodiment 27

A method of treatment of pain or cancer in a mammal, comprising treating said mammal with an effective amount of a compound of the formula (I) or a pharmaceutically acceptable salt thereof, as defined in any one of embodiments 1 to 19.

### Embodiment 28

A compound or salt according to any one of embodiments 1 to 19 for use in a medical treatment in combination with a further drug substance.

Further embodiments include:

Any novel genus of intermediates described in the Schemes below;

Any novel specific intermediate described in the Preparations below;

Any novel process described herein.

“Halogen” means a fluoro, chloro, bromo or iodo group.

“Alkyl” groups, containing the requisite number of carbon atoms, can be unbranched or branched. Examples of alkyl include methyl, ethyl, n-propyl, i-propyl, n-butyl, i-butyl, sec-butyl and t-butyl.

“Pharmaceutically acceptable salts” of the compounds of formula I include the acid addition and base addition salts (including disalts, hemisalts, etc.) thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base addition salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, Weinheim, Germany, 2002).

The compounds of the invention include compounds of formula I and salts thereof as hereinbefore defined, polymorphs, and isomers thereof (including optical, geometric and tautomeric isomers) as hereinafter defined and isotopically-labelled compounds of formula I.

Unless otherwise specified, compounds of formula (I) containing one or more asymmetric carbon atoms can exist as two or more stereoisomers. Where a compound of formula (I) contains for example, a keto or guanidine group or an aromatic moiety, tautomeric isomerism (‘tautomerism’) can occur. It follows that a single compound may exhibit more than one type of isomerism.

Included within the scope of the claimed compounds of the present invention are all stereoisomers, geometric isomers and tautomeric forms of the compounds of formula (I), including compounds exhibiting more than one type of isomerism, and mixtures of one or more thereof. Also included are acid addition or base addition salts wherein the counterion is optically active, for example, D-lactate or L-lysine, or racemic, for example, DL-tartrate or DL-arginine.

Examples of types of potential tautomerisms shown by the compounds of the invention include hydroxypyridine [symbol] pyridone; amide [symbol] hydroxyl-imine and keto [symbol] enol tautomersims:

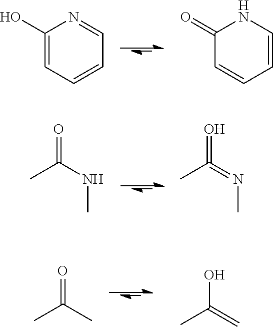

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or other derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on a resin with an asymmetric stationary phase and with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture.

Mixtures of stereoisomers may be separated by conventional techniques known to those skilled in the art. [see, for example, “Stereochemistry of Organic Compounds” by E L Eliel (Wiley, New York, 1994).]

The present invention includes all pharmaceutically acceptable isotopically-labelled compounds of formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds of formula (I) can generally be prepared by conventional techniques known to those skilled in the art or by processes analogous to those described in the accompanying Examples and Preparations using an appropriate isotopically-labelled reagents in place of the non-labelled reagent previously employed

The routes below, including those mentioned in the Examples and Preparations, illustrate methods of synthesising compounds of formula (I). The skilled person will appreciate that the compounds of the invention, and intermediates thereto, could be made by methods other than those specifically described herein, for example by adaptation of the methods described herein, for example by methods known in the art. Suitable guides to synthesis, functional group interconversions, use of protecting groups, etc., are for example:“Comprehensive Organic Transformations” by R C Larock, VCH Publishers Inc. (1989); Advanced Organic Chemistry” by J. March, Wiley Interscience (1985); “Designing Organic Synthesis” by S Warren, Wiley Interscience (1978); “Organic Synthesis—The Disconnection Approach” by S Warren, Wiley Interscience (1982); “Guidebook to Organic Synthesis” by R K Mackie and DM Smith, Longman (1982); “Protective Groups in Organic Synthesis” by T W Greene and P G M Wuts, John Wiley and Sons, Inc. (1999); and “Protecting Groups” by P J, Kocienski, Georg Thieme Verlag (1994); and any updated versions of said standard works.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

In the general synthetic methods below, unless otherwise specified, the substituents are as defined above with reference to the compounds of formula (I) above.

Where ratios of solvents are given, the ratios are by volume.

### General Schemes

The compounds of the invention may be prepared by any method known in the art for the preparation of compounds of analogous structure. In particular, the compounds of the invention can be prepared by the procedures described by reference to the Schemes that follow, or by the specific methods described in the Examples, or by similar processes to either.

The skilled person will appreciate that the experimental conditions set forth in the schemes that follow are illustrative of suitable conditions for effecting the transformations shown, and that it may be necessary or desirable to vary the precise conditions employed for the preparation of compounds of formula (I). It will be further appreciated that it may be necessary or desirable to carry out the transformations in a different order from that described in the schemes, or to modify one or more of the transformations, to provide the desired compound of the invention.

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino or carboxylic acid groups. The protecting groups used in the preparation of the compounds of the invention may be used in conventional manner. See, for example, those described in ‘Greene's Protective Groups in Organic Synthesis’ by Theodora W Greene and Peter G M Wuts, third edition, (John Wiley and Sons, 1999), in particular chapters 7 (“Protection for the Amino Group”) and 5 (“Protection for the Carboxyl Group”), incorporated herein by reference, which also describes methods for the removal of such groups.

All of the compounds of the formula (I) can be prepared by the procedures described in the general methods presented below or by routine modifications thereof. The present invention also encompasses any one or more of these processes for preparing the derivatives of formula (I), in addition to any novel intermediates used therein.

According to a first process, compounds of formula (I) may be prepared from compounds of formula (V) and (IV) as illustrated by Scheme 1,

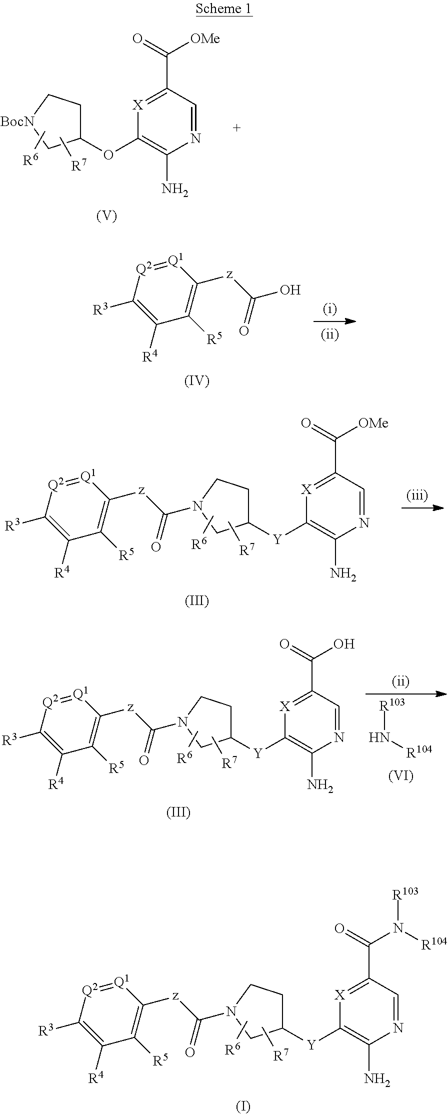

Compounds of formulae (IV) and (VI) are commercially available or may be synthesized by those skilled in the art according to the literature or preparations described herein. Compounds of formula (V) may be synthesized according to Scheme 4.

Racemic compounds of formula (I) may be chirally separated into their respective enantiomers using appropriate chiral chromatography.

Compounds of formula (I) may be prepared from compounds of formula (II) according to process step (ii), an amide bond forming step with compounds of formula (VI) with activation of the carboxylic acid (II), using a suitable organic base and suitable coupling agents such as EDCl/HOBt or HATU. Preferred conditions comprise HATU in DMF or pyridine either with or without triethylamine or EDCI with HOBT in DCM with triethylamine, both at room temperature.

Compounds of formula (II) may be prepared from compounds of formula (III) according to process step (iii), a base-mediated hydrolysis reaction using an inorganic base either at room or elevated temperatures. Preferred conditions comprise aqueous sodium hydroxide in methanol at 60° C. or lithium hydroxide in THF and water at room temperature.

Compounds of formula (III) may be prepared from compounds of formula (IV) and (V) according to process step (i), an acid mediated deprotection reaction followed by process step (ii) and amide bond forming reaction as described above. Preferred deprotection conditions comprise 4M HCl in dioxane or neat TFA, both at room temperature.

According to a second process, compounds of formula (I) may be prepared from compounds of formula (V) and (IV) as illustrated by Scheme 2,

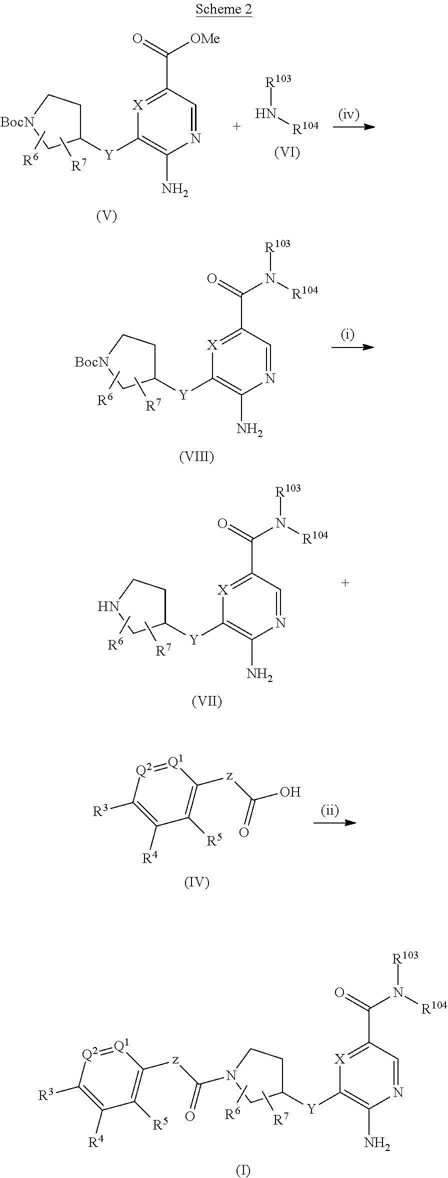

Compounds of formulae (IV) and (VI) are commercially available or may be synthesized by those skilled in the art according to the literature or preparations described herein. Compounds of formula (V) may be synthesized according to Scheme 4.

Compounds of formula (I) may be prepared from compounds of formula (VII) according to process step (ii), an amide bond forming step with compounds of formula (IV) as described in Scheme 1.

Compounds of formula (VII) may be prepared from compounds of formula (VIII) according to process step (i), an acid mediated deprotection step as described in Scheme 1.

Compounds of formula (VIII) may be prepared from compounds of formulae (V) and (VI) according to process step (iv), a substitution reaction with solutions of compounds of formula (VI) in methanol. Preferred conditions comprise solutions of compounds of formula (VI) in methanol at elevated temperatures of 60° C. in a sealed vessel.

According to a third process, compounds of formula (VIII) may be prepared from compounds of formula (IX) as illustrated by Scheme 3,

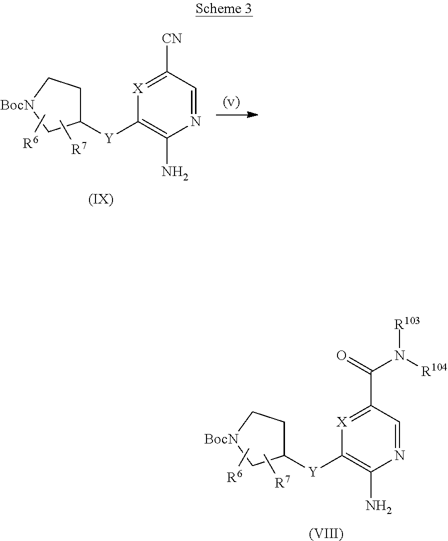

Wherein R¹⁰³ and R¹⁰⁴ are H;

Compounds of formula (IX) may be prepared as described in Scheme 4. Compounds of formula (VIII) may be prepared from compounds of formula (IX) according to process step (v), a functional group interconversion of a nitrile to an amide under oxidative conditions. Preferred conditions comprise lithium hydroxide with hydrogen peroxide in methanol at from 0° C. to room temperature.

According to a fourth process, compounds of formula (IX) and (V) may be prepared from compounds of formula (XI) as illustrated by Scheme 4,

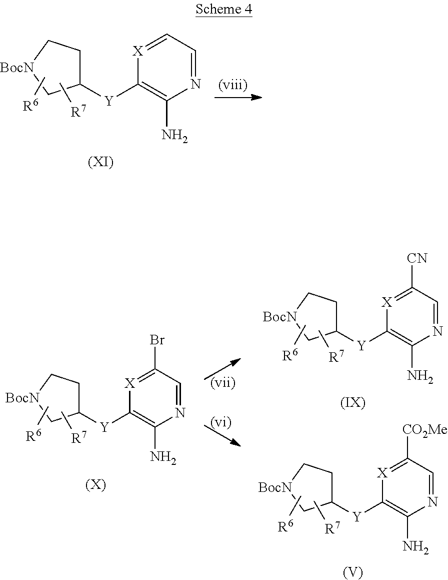

Compounds of formula (XI) may be prepared as described in Schemes 5-7. Compounds of formula (V) may be prepared from compounds of formula (X) according to process step (vi), a carbonylation reaction catalysed by a suitable palladium catalyst with suitable phosphine ligands in an alcoholic solvent at elevated temperatures under an atmosphere of carbon monoxide. Preferred conditions comprise Pd(dppf)Cl₂ with triethylamine in methanol under 80 psi carbon monoxide at 80° C., or alternatively Pd(OAc)₂ with DIPEA or triethylamine and dppp or dppf in methanol under a balloon of carbon monoxide at between 80-100° C.

Compounds of formula (IX) may be prepared from compounds of formula (X) according to process step (vii), a cyanation reaction catalysed by a suitable palladium catalyst with suitable phosphine ligands and a transition metal cyanide at elevated temperatures. Preferred conditions comprise Pd(dba)₂ with dppf and zinc cyanide in DMF at 100° C. Compounds of formula (X) may be prepared from compounds of formula (XI) according to process step (viii) an electrophilic bromination reaction. Preferred conditions comprise N-bromosuccinimide in MeCN at from 0° C. to room temperature.

According to a fifth process, compounds of formula (XI) may be prepared from compounds of formula (XIV) and (XV) as illustrated by Scheme 5,

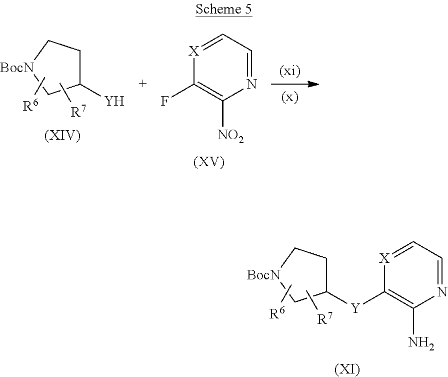

Wherein Y is O or CH₂O;

Compounds of formulae (XIV) and (XV) are commercially available or may be synthesized by those skilled in the art according to the literature or preparations described herein.

Compounds of formula (XI) may be prepared from compounds of formula (XIV) and (XV) according to process step (xi), a nucleophilic aromatic substitution reaction followed by process step (x), a reduction reaction. Preferred conditions comprise cesium carbonate in THF at elevated temperatures of 65° C. followed by 10% palladium on carbon under hydrogenation in either IMS or MeOH at 50 psi hydrogen at room temperature.

According to a sixth process, compounds of formula (XI) may be prepared from compounds of formula (XII) and (XIII) as illustrated by Scheme 6,

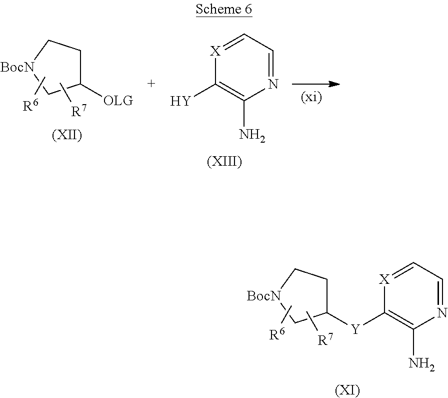

Wherein Y is O or OCH₂ and wherein LG is a leaving group such as mesylate; Compounds of formulae (XII) and (XIII) are commercially available or may be synthesized by those skilled in the art according to the literature or preparations described herein.

Compounds of formula (XI) may be prepared from compounds of formula (XII) and (XIII) according to process step (xi), a nucleophilic substitution reaction. Preferred conditions comprise cesium carbonate in DMF at 130° C.

According to a seventh process, compounds of formula (XI) may be prepared from compounds of formula (XVI) and (XVII) as illustrated by Scheme 7,

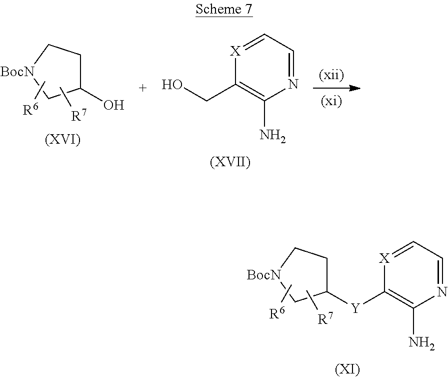

Wherein Y is CH₂O;

Compounds of formulae (XVI) and (XVII) are commercially available or may be synthesized by those skilled in the art according to the literature or preparations described herein.

Compounds of formula (XI) may be prepared from compounds of formula (XVI) and (XVII) according to process step (xii), a halogenation reaction with compounds of formula (XVII) followed by process step (xi), a nucleophilic substitution reaction with compounds of formula (XVI), with conditions as described in Scheme 6. Preferred halogenation conditions comprise thionyl chloride in THF at 0° C., and preferred substitution conditions use by sodium hydride in THF at from 0° C. to elevated temperatures of 60° C.

According to a further embodiment the present invention provides novel intermediate compounds described herein.

Pharmaceutically acceptable salts of a compound of formula (I) may be readily prepared by mixing together solutions of the compound of formula (I) and the desired acid or base, as appropriate. The salt may precipitate from solution and be collected by filtration or may be recovered by evaporation of the solvent. The degree of ionisation in the salt may vary from completely ionised to almost non-ionised.

A trk antagonist may be usefully combined with another pharmacologically active compound, or with two or more other pharmacologically active compounds, particularly in the treatment of pain. The skilled person will appreciate that such combinations offer the possibility of significant advantages, including patient compliance, ease of dosing and synergistic activity.

In the combinations that follow the compound of the invention may be administered simultaneously, sequentially or separately in combination with the other therapeutic agent or agents.

A trk antagonist compound of formula (I), or a pharmaceutically acceptable salt thereof, as defined above, may be administered in combination with one or more agents selected from:

a selective Nav1.3 channel modulator, such as a compound disclosed in WO2008/118758;
a selective Nav1.7 channel modulator, such as a compound disclosed in WO2010/079443, e.g. 4-[2-(5-amino-1H-pyrazol-4-yl)-4-chlorophenoxy]-5-chloro-2-fluoro-N-1,3-thiazol-4-ylbenzenesulfonamide or 4-[2-(3-amino-1H-pyrazol-4-yl)-4-(trifluoromethyl)phenoxy]-5-chloro-2-fluoro-N-1,3-thiazol-4-ylbenzenesulfonamide, or a pharmaceutically acceptable salt of either;
a selective Nav1.8 channel modulator;
a selective Nav1.9 channel modulator;
a compound which modulates activity at more than one Nav channel, including a non-selective modulator such as bupivacaine, carbamazepine, lamotrigine, lidocaine, mexiletine or phenytoin;
any inhibitor of nerve growth factor (NGF) signaling, such as: an agent that binds to NGF and inhibits NGF biological activity and/or downstream pathway(s) mediated by NGF signaling (e.g. tanezumab), a TrkA antagonist or a p75 antagoinsist, or an agent that inhibits downstream signaling in regard to NGF stimulated TrkA or P75 signalling; a compound which increases the levels of endocannabinoid, such as a compound with fatty acid amid hydrolase inhibitory (FAAH) or monoacylglycerol lipase (MAGL) activity; an analgesic, in particular paracetamol;
an opioid analgesic, such as: buprenorphine, butorphanol, cocaine, codeine, dihydrocodeine, fentanyl, heroin, hydrocodone, hydromorphone, levallorphan levorphanol, meperidine, methadone, morphine, nalmefene, nalorphine, naloxone, naltrexone, nalbuphine, oxycodone, oxymorphone, propoxyphene or pentazocine; an opioid analgesic which preferentially stimulates a specific intracellular pathway, for example G-protein as opposed to beta arrestin recruitment, such as TRV130; an opioid analgesic with additional pharmacology, such as: noradrenaline (norepinephrine) reuptake inhibitory (NRI) activity, e.g. tapentadol; serotonin and norepinephrine reuptake inhibitory (SNRI) activity, e.g. tramadol; or nociceptin receptor (NOP) agonist activity, such as GRT6005;
a nonsteroidal antiinflammatory drug (NSAID), such as a non-selective cyclooxygenase (COX) inhibitor, e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac; or a COX-2 selective inhibitor, e.g. celecoxib, deracoxib, etoricoxib, mavacoxib or parecoxib;
a prostaglandin E₂ subtype 4 (EP4) antagonist;
a microsomal prostaglandin E synthase type 1 (mPGES-1) inhibitor;
a sedative, such as glutethimide, meprobamate, methaqualone or dichloralphenazone;
a GABA_{A} modulator with broad subtype modulatory effects mediated via the benzodiazepine binding site, such as chlordiazepoxide, alprazolam, diazepam, lorazepam, oxazepam, temazepam, triazolam, clonazepam or clobazam;
a GABA_{A} modulator with subtype-selective modulatory effects mediated via the benzodiazepine binding site with reduced adverse effects, for example sedation, such as TPA023, TPA023B, L-838,417, CTP354 or NSD72;
a GABA_{A} modulator acting via alternative binding sites on the receptor, such as barbiturates, e.g. amobarbital, aprobarbital, butabital, mephobarbital, methohexital, pentobarbital, phenobartital, secobarbital, or thiopental; neurosteroids such as alphaxalone, alphadolone or ganaxolone; β-subunit ligands, such as etifoxine; or δ-preferring ligands, such as gaboxadol;
a GlyR3 agonist or positive allosteric modulator;
a skeletal muscle relaxant, e.g. baclofen, carisoprodol, chlorzoxazone, cyclobenzaprine, metaxolone, methocarbamol or orphrenadine;
a glutamate receptor antagonist or negative allosteric modulator, such as an NMDA receptor antagonist, e.g. dextromethorphan, dextrorphan, ketamine or, memantine; or an mGluR antagonist or modulator;
an alpha-adrenergic, such as clonidine, guanfacine or dexmetatomidine;
a beta-adrenergic such as propranolol;
a tricyclic antidepressant, e.g. desipramine, imipramine, amitriptyline or nortriptyline;
a tachykinin (NK) antagonist, such as aprepitant or maropitant; a muscarinic antagonist, e.g oxybutynin, tolterodine, propiverine, tropsium chloride, darifenacin, solifenacin, temiverine and ipratropium;
a Transient Receptor Potential V1 (TRPV1) receptor agonist (e.g. resinferatoxin or capsaicin) or antagonist (e.g. capsazepine or mavatrap);
a Transient Receptor Potential A1 (TRPA1) receptor agonist (e.g. cinnamaldehyde or mustard oil) or antagonist (e.g. GRC17536 or CB-625);
a Transient Receptor Potential M8 (TRPM8) receptor agonist (e.g. menthol or icilin) or antagonist;
a Transient Receptor Potential V3 (TRPV3) receptor agonist or antagonist (e.g. GRC-15300);
a corticosteroid such as dexamethasone;
a 5-HT receptor agonist or antagonist, particularly a 5-HT_{1B/1D} agonist, such as eletriptan, sumatriptan, naratriptan, zolmitriptan or rizatriptan;
a 5-HT_{2A} receptor antagonist;
a cholinergic (nicotinic) analgesic, such as ispronicline (TC-1734), vareniclineor nicotine;
a PDEV inhibitor, such sildenafil, tadalafilor vardenafil;
an alpha-2-delta ligand such as gabapentin, gabapentin enacarbil or pregabalin;
a serotonin reuptake inhibitor (SRI) such as sertraline, demethylsertraline, fluoxetine, norfluoxetine, fluvoxamine, paroxetine, citalopram, desmethylcitalopram, escitalopram, d,l-fenfluramine, femoxetine, ifoxetine, cyanodothiepin, litoxetine, dapoxetine, nefazodone, cericlamine and trazodone;
anNRI, such as maprotiline, lofepramine, mirtazepine, oxaprotiline, fezolamine, tomoxetine, mianserin, buproprion, buproprion metabolite hydroxybuproprion, nomifensine and viloxazine, especially a selective noradrenaline reuptake inhibitor such as reboxetine;
an SNRI, such as venlafaxine, 0-desmethylvenlafaxine, clomipramine, desmethylclomipramine, duloxetine, milnacipran and imipramine;
an inducible nitric oxide synthase (iNOS) inhibitor;
a leukotriene B4 antagonist;
a 5-lipoxygenase inhibitor, such as zileuton;
a potassium channel opener or positive modulator, such as an opener or positive modulator of KCNQ/Kv7 (e.g. retigabine or flupirtine), a G protein-coupled inwardly-rectifying potassium channel (GIRK), a calcium-activated potassium channel (Kca) or a potassium voltage-gated channel such as a member of subfamily A (e.g. Kv1.1), subfamily B (e.g. Kv2.2) or subfamily K (e.g. TASK, TREK or TRESK);
a P2X₃ receptor antagonist (e.g. AF219) or an antagonist of a receptor which contains as one of its subunits the P2X₃ subunit, such as a P2X_{2/3} heteromeric receptor;
a Ca_{V}2.2 calcium channel blocker (N-type), such as ziconotide; and
a Ca_{V}3.2 calcium channel blocker (T-type), such as ethosuximide.

Pharmaceutical compositions suitable for the delivery of compounds and salts of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

Compounds and salts of the invention intended for pharmaceutical use may be prepared and administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

### Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations, such as tablets, capsules containing particulates, liquids, or powders; lozenges (including liquid-filled), chews; multi- and nano-particulates; gels, solid solution, liposome, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavoring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tableting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed above may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

### Parenteral Administration

The compounds and salts of the invention may be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) and salts used in the preparation of parenteral solutions may be increased by the use of appropriate formulation techniques, such as the incorporation of solubility-enhancing agents.

Formulations for parenteral administration may be formulated to be immediate and/or modified release. Thus, compounds and salts of the invention may be formulated as a solid, semi-solid, or thixotropic liquid for administration as an implanted depot providing modified release of the active compound. An example of such formulations include drug-coated stents.

### Topical Administration

The compounds and salts of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, Finnin and Morgan, J Pharm Sci, 88 (10), 955-958 (October 1999).] Other means of topical administration include delivery by electroporation, iontophoresis, phonophoresis, sonophoresis and microneedle or needle-free (e.g. Powderject™, Bioject™, etc.) injection.

### Inhaled/Intranasal Administration

The compounds and salts of the invention may also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

A pressurised container, pump, spray, atomizer, or nebuliser may contain a solution or suspension of the compound(s) or salt(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or HPMC), blisters and cartridges for use in an inhaler or insufflator may be formulated to contain a powder mix of the compound or salt of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound or salt of the invention per actuation and the actuation volume may vary from 1 μl to 100 μl. A typical formulation may comprise a compound of formula (I) or salt thereof, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by a prefilled capsule, blister or pocket or by a system that utilises a gravimetrically fed dosing chamber. Units in accordance with the invention are typically arranged to administer a metered dose or “puff” containing from 1 to 5000 μg of the compound or salt. The overall daily dose will typically be in the range 1 μg to 20 mg which may be administered in a single dose or, more usually, as divided doses throughout the day.

### Rectal/Intravaginal Administration

The compounds and salts of the invention may be administered rectally or vaginally, for example, in the form of a suppository, pessary, or enema. Cocoa butter is a traditional suppository base, but various well known alternatives may be used as appropriate.

### Ocular and Aural Administration

The compounds and salts of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid; a cellulosic polymer, for example, hydroxypropylmethylcellulose, hydroxyethylcellulose, or methyl cellulose; or a heteropolysaccharide polymer, for

example, gelan gum, may be incorporated together with a preservative, such as benzalkonium chloride. Such formulations may also be delivered by iontophoresis.

### Other Technologies

The compounds and salts of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds and salts of the invention is typically in the range 0.1 mg to 200 mg depending, of course, on the mode of administration, preferred in the range 1 mg to 100 mg and more preferred in the range 1 mg to 50 mg. The total daily dose may be administered in single or divided doses.

These dosages are based on an average human subject having a weight of about 65 kg to 70 kg. The physician will readily be able to determine doses for subjects whose weight falls outside this range, such as infants and the elderly.

For the above-mentioned therapeutic uses, the dosage administered will, of course, vary with the compound or salt employed, the mode of administration, the treatment desired and the disorder indicated. The total daily dosage of the compound of formula (I)/salt/solvate (active ingredient) will, generally, be in the range from 1 mg to 1 gram, preferably 1 mg to 250 mg, more preferably 10 mg to 100 mg. The total daily dose may be administered in single or divided doses. The present invention also encompasses sustained release compositions.

The pharmaceutical composition may, for example, be in a form suitable for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefor, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

For parenteral dosages, this may conveniently be prepared as a solution or as a dry powder requiring dissolution by a pharmacist, medical practitioner or the patient. It may be provided in a bottle or sterile syringe. For example it may be provided as a powder in a multicompartment syringe which allows the dry powder and solvent to be mixed just prior to administration (to aid long-term stability and storage). Syringes could be used which allow multiple doses to be administered from a single device.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations as discussed below. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

A composition of the present invention can be administered by a variety of methods known in the art. The route and/or mode of administration vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, ed., Marcel Dekker, Inc., New York, (1978). Pharmaceutical compositions are preferably manufactured under GMP conditions.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

The precise dosage administered of each active ingredient will vary depending upon any number of factors, including but not limited to, the type of animal and type of disease state being treated, the age of the animal, and the route(s) of administration.

The following non-limiting Preparations and Examples illustrate the preparation of compounds and salts of the present invention.

In the non-limiting Examples and Preparations that are set out later in the description, and in the aforementioned Schemes, the following the abbreviations, definitions and analytical procedures may be referred to:
t-Bu₃PHBF₄ is tri-tert-butylphosphinetetrafluoroborate salt; t-BuOH is tert-butanol; ° C. is degrees centigrade; COMU® is (1-cyano-2-ethoxy-2-oxoethylideneaminooxy)dimethylamino-morpholino-carbenium hexafluorophosphate; Cs₂CO₃ is cesium carbonate; CuSO₄.5H₂O is copper sulphate pentahydrate; DCM is dichloromethane; methylene chloride; DEA is diethylamine; DIPEA is N-ethyldiisopropylamine, N,N-diisopropylethylamine; DMF is N,N-dimethylformamide; DMSO is dimethyl sulfoxide; EDCI is 1-(3-dimethylaminopropyl)-3-ethylcarbodiimide hydrochloride; EtOAc is ethyl acetate; EtOH is ethanol; H₂SO₄ is sulphuric acid; HATU is 1-[Bis(dimethylamino)methylene]-1H-1,2,3-triazolo[4,5-b]pyridinium 3-oxid hexafluorophosphate; HCl is hydrochloric acid; HOBt is hydroxybenzotriazole; HPLC is high-performance liquid chromatography; IPA is isopropanol; KOH is potassium hydroxide; KOAc is potassium acetate; LCMS is liquid chromatography mass spectrometry (Rₜ=retention time); Me is methyl; MeCN is acetonitrile; MeOH is methanol; MgSO₄ is magnesium sulphate; MS is mass spectrometry; NaHCO₃ is sodium hydrogen carbonate; NaOH is sodium hydroxide; Na₂SO₄ is sodium sulphate; NH₃ is ammonia; Pd/C is palladium on carbon; Pd(PPh₃)₄ is palladium tetrakis; PdCl₂(PPh₃)₂ is bis(triphenylphosphine)palladium (II) dichloride; Pd₂(dba)₃ is tris(dibenzylideneacetone)dipalladium (0); Pd(dppf)₂Cl₂ is [1,1′-bis(diphenylphosphino)ferrocene]dichloropalladium(II), complex with dichloromethane; SEM is 2-[(trimethylsilyl)ethoxy]methyl; TFA is trifluoroacetate; THF is tetrahydrofuran; THP is tetrahydropyran and TLC is thin layer chromatography;

¹H and ¹⁹F Nuclear magnetic resonance (NMR) spectra were in all cases consistent with the proposed structures. Characteristic chemical shifts (6) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionisation (ESI) or atmospheric pressure chemical ionisation (APCI).

Where relevant and unless otherwise stated the m/z data provided are for isotopes ¹⁹F, ³⁵Cl, ⁷⁹Br and ¹²⁷I.

### Preparative HPLC:

Where singleton compounds are purified by preparative HPLC, there are two methods used, shown below:

| | |
|---|---|
| Method 1 | acidic conditions |
| Column | Gemini NX C18, 5 um 21.2 × 100 mm |
| Temperature | Ambient |
| Detection | ELSD-MS |
| Mobile Phase A | 0.1% formic acid in water |
| Mobile Phase B | 0.1% formic acid in acetonitrile |
| Gradient initial | 0% B, 1 mins-5% B; 7 mins-98% B; |
| | 9 mins-98% B; 9.1 mins-5% B; |
| | 10 mins-5% B |
| Flow rate | 18 mL/min |
| Injection volume | 1000 uL |

| | |
|---|---|
| Method 2 | basic conditions |
| Column | Gemini NX C18, 5 um 21.2 × 100 mm |
| Temperature | Ambient |
| Detection | ELSD-MS |
| Mobile Phase A | 0.1% diethylamine in water |
| Mobile Phase B | 0.1% diethylamine in acetonitrile |
| Gradient initial | 0% B, 1 mins-5% B; 7 mins-98% B; |
| | 9 mins-98% B; 9.1 mins-5% B; |
| | 10 mins-5% B |
| Flow rate | 18 mL/min |
| Injection volume | 1000 uL |

### Example 1

### (S)-6-amino-5-[(4,4-difluoro-1-{[4-(trifluoromethoxy)phenyl]acetyl}pyrrolidin-3-yl)oxy]-N-methylpyridine-3-carboxamide

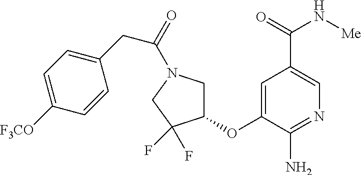 [SMILES: CNC(=O)c1cnc(N)c(O[C@H]2CN(C(=O)Cc3ccc(OC(F)(F)F)cc3)CC2(F)F)c1]

To a solution of (S)-6-amino-5-[(4,4-difluoro-1-{[4-(trifluoromethoxy)phenyl]acetyl}pyrrolidin-3-yl)oxy]pyridine-3-carboxylic acid (Preparation 1, 424 mg, 0.92 mmol) in DMF (3 mL) was added methylamine hydrochloride (93 mg, 1.38 mmol), triethylamine (0.51 mL, 3.68 mmol) and HATU (535 mg, 1.38 mmol). The reaction was stirred at room temperature for 30 minutes before diluting with water (15 mL) and extracting into EtOAc (3×15 mL). The organic extracts were combined, washed with water (15 mL), 10% aqueous potassium carbonate solution (2×15 mL), brine (15 mL) and concentrated in vacuo. To the residue was added 2M methylamine in THF (2 mL) and the reaction was stirred at room temperature for 10 minutes. The reaction was concentrated in vacuo and dissolved in EtOAc. The solution was washed with 10% aqueous potassium carbonate solution (2×15 mL), brine (15 mL), dried over sodium sulphate and concentrated in vacuo. The residue was purified using silica gel column chromatography eluting with 2-10% MeOH in DCM followed by trituration with TBME to afford the title compound as a white solid (170 mg, 39%)_{.}

¹H NMR (400 MHz, DMSO-d₆): δ ppm 2.40-2.50 (m, 2H), 2.70 (d, 3H), 3.60-4.40 (m, 4H), 5.20-5.30 (m, 1H), 6.38-6.45 (m, 2H), 7.20-7.40 (m, 4H), 7.60 (s, 1H), 8.05-8.20 (m, 2H).

¹⁹F NMR (400 MHz, DMSO-d₆): δ ppm −56.5 (s, 3F), −106 (m, 1F), −121 (m, 1F).

MS m/z 475 [M+H]⁺

The title compound may also be prepared from chiral separation of the racemate prepared from racemic-6-amino-5-[(4,4-difluoro-1-{[4-(trifluoromethoxy)phenyl]acetyl}pyrrolidin-3-yl)oxy]pyridine-3-carboxylic acid (Preparation 3). The residue was separated into the two enantiomers using chiral column chromatography according to the conditions described below:

Column: Chiralcel OJ-H 250×4.6 mm

Mobile phase: 10% MeOH in H₂O

Flow rate: 3 mL/min.

Rt=3.60 minutes and 5.22 minutes

The two enantiomers were arbitrarily assigned stereochemistry:

### Example 1

(S)-6-amino-5-[(4,4-difluoro-1-{[4-(trifluoromethoxy)phenyl]acetyl}pyrrolidin-3-yl)oxy]-N-methylpyridine-3-carboxamide

### Example 2

(R)-6-amino-5-[(4,4-difluoro-1-{[4-(trifluoromethoxy)phenyl]acetyl}pyrrolidin-3-yl)oxy]-N-methylpyridine-3-carboxamide

### Examples 3 and 4 (S) and (R)-6-amino-5-{[(4,4-difluoro-1-{[4-(trifluoromethoxy)phenyl]acetyl}pyrrolidin-3-yl)oxy]methyl}-N-methylpyridine-3-carboxamide

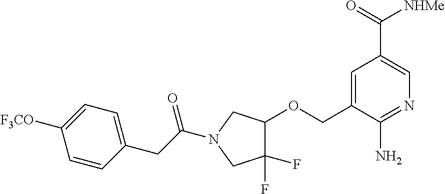 [SMILES: CNC(=O)c1cnc(N)c(COC2CN(C(=O)Cc3ccc(OC(F)(F)F)cc3)CC2(F)F)c1]

The racemic title compound was prepared according to the method described for Example 1 using racemic-6-amino-5-{[(4,4-difluoro-1-{[4-(trifluoromethoxy)phenyl]acetyl}pyrrolidin-3-yl)oxy]methyl}pyridine-3-carboxylic acid (Preparation 4) and methylamine.

The residue was separated into the two enantiomers using chiral column chromatography according to the conditions described below:

Column: Chiralpak AD-3 4.6×100 mm, 3 micron

Mobile phase: 0.1% DEA/MeOH in water

Flow rate: 4 mL/min.

Rt=0.957 minutes and 1.758 minutes

¹H NMR (400 MHz, DMSO-d₆): δ ppm 2.71-2.79 (m, 3H), 3.66-3.78 (m, 4H), 3.93-4.19 (m, 2H), 4.26-4.42 (br s, 1H), 4.59 (s, 1H), 4.61 (s, 1H), 6.34 (br s, 2H), 7.27-7.37 (m, 4H), 7.85 (d, 1H), 8.16 (br s, 1H), 8.45 (d, 1H).

MS m/z 489 [M+H]⁺

The two enantiomers were arbitrarily assigned stereochemistry:

### Example 3

(R)-6-amino-5-{[(4,4-difluoro-1-{[4-(trifluoromethoxy)phenyl]acetyl}pyrrolidin-3-yl)oxy]methyl}-N-methylpyridine-3-carboxamide

### Example 4

(S)-6-amino-5-{[(4,4-difluoro-1-{[4-(trifluoromethoxy)phenyl]acetyl}pyrrolidin-3-yl)oxy]methyl}-N-methylpyridine-3-carboxamide

### Examples 5 and 6

### (S) and (R)-6-amino-5-{[(4,4-difluoro-1-{[4-(trifluoromethoxy)phenyl]acetyl}pyrrolidin-3-yl)oxy]methyl}pyridine-3-carboxamide

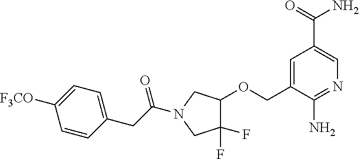 [SMILES: NC(=O)c1cnc(N)c(COC2CN(C(=O)Cc3ccc(OC(F)(F)F)cc3)CC2(F)F)c1]

The racemic title compound was prepared according to the method described for Example 1 using racemic-6-amino-5-{[(4,4-difluoro-1-{[4-(trifluoromethoxy)phenyl]acetyl}pyrrolidin-3-yl)oxy]methyl}pyridine-3-carboxylic acid (Preparation 4) and ammonium chloride.

The residue was separated into the two enantiomers using chiral column chromatography according to the conditions described below:

Column: Chiralpak AS-H 4.6×150 mm, 5 micron

Mobile phase: 25% MeOH in water with 0.1% DEA

Flow rate: 4 mL/min.

Rt=1.032 minutes and 1.750 minutes

¹H NMR (400 MHz, DMSO-d₆): δ ppm 3.59-3.88 (m, 4H), 3.88-4.17 (m, 2H), 4.24-4.47 (m, 1H), 4.59 (d, 2H), 6.36 (br s, 2H), 7.05 (br s, 1H), 7.22-7.41 (m, 4H), 7.70 (br s, 1H), 7.88 (d, 1H), 8.41-8.56 (m, 1H).

MS m/z 475 [M+H]⁺

The two enantiomers were arbitrarily assigned stereochemistry:

### Example 5

(R)-6-amino-5-{[(4,4-difluoro-1-{[4-(trifluoromethoxy)phenyl]acetyl}pyrrolidin-3-yl)oxy]methyl}pyridine-3-carboxamide

### Example 6

(S)-6-amino-5-{[(4,4-difluoro-1-{[4-(trifluoromethoxy)phenyl]acetyl}pyrrolidin-3-yl)oxy]methyl}pyridine-3-carboxamide

### Examples 7 and 8

### (S) and (R)-6-amino-5[(4,4-difluoro-1-{[4-(trifluoromethoxy)phenyl]acetyl}pyrrolidin-3-yl)oxy]-N-(2-hydroxyethyl)pyridine-3-carboxamide

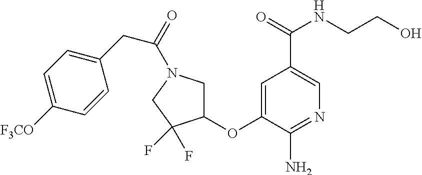 [SMILES: Nc1ncc(C(=O)NCCO)cc1OC1CN(C(=O)Cc2ccc(OC(F)(F)F)cc2)CC1(F)F]

The racemic title compound was prepared according to the method described for Example 1 using racemic-6-amino-5-[(4,4-difluoro-1-{[4-(trifluoromethoxy)phenyl]acetyl}pyrrolidin-3-yl)oxy]pyridine-3-carboxylic acid (Preparation 3) and ethanolamine.

The residue was separated into the two enantiomers using chiral column chromatography according to the conditions described below:

Column: Lux-Cellulose-1 4.6×250 mm, 5 micron

Mobile phase: 40% MeOH in water

Flow rate: 3 mL/min.

Rt=1.95 minutes and 2.61 minutes

¹H NMR (400 MHz, DMSO-d₆): δ ppm 3.45-3.56 (m, 2H), 3.65-3.87 (m, 3H), 3.94 (d, 1H), 4.11 (br s, 1H), 4.17-4.63 (m, 1H), 4.71 (br s, 1H), 5.18-5.38 (m, 1H), 6.43 (s, 1H), 6.40 (s, 1H), 7.23-7.44 (m, 4H), 7.62 (br s, 1H), 8.16 (d, 1H), 8.20 (br s, 1H).

MS m/z 505 [M+H]⁺

The two enantiomers were arbitrarily assigned stereochemistry:

### Example 7

(R)-6-amino-5-[(4,4-difluoro-1-{[4-(trifluoromethoxy)phenyl]acetyl}pyrrolidin-3-yl)oxy]-N-(2-hydroxyethyl)pyridine-3-carboxamide

### Example 8

(S)-6-amino-5-[(4,4-difluoro-1-{[4-(trifluoromethoxy)phenyl]acetyl}pyrrolidin-3-yl)oxy]-N-(2-hydroxyethyl)pyridine-3-carboxamide

### Examples 9 and 10

### (S) and (R)-6-amino-5-[(4,4-difluoro-1-{[4-(trifluoromethoxy)phenyl]acetyl}pyrrolidin-3-yl)oxy]-N-(2-hydroxy-2-methylpropyl)pyridine-3-carboxamide

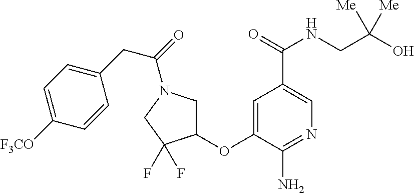 [SMILES: CC(C)(O)CNC(=O)c1cnc(N)c(OC2CN(C(=O)Cc3ccc(OC(F)(F)F)cc3)CC2(F)F)c1]

The racemic title compound was prepared according to the method described for Example 1 using racemic-5-[(4,4-difluoro-1-{[4-(trifluoromethoxy)phenyl]acetyl}pyrrolidin-3-yl)oxy]pyridine-3-carboxylic acid (Preparation 3) and 1-amino-2-methylpropan-2-ol.

The residue was separated into the two enantiomers using chiral column chromatography according to the conditions described below:

Column: Lux-Cellulose-1 4.6×250 mm, 5 micron

Mobile phase: 40% MeOH in water

Flow rate: 3 mL/min.

Rt=1.677 minutes and 2.109 minutes

¹H NMR (400 MHz, DMSO-d₆): δ ppm 1.10 (d, 6H), 3.18-3.27 (m, 2H), 3.65-3.85 (m, 3H), 3.86-3.99 (m, 1H), 4.01-4.17 (m, 1H), 4.17-4.48 (m, 1H), 4.54 (d, 1H), 5.23-5.41 (m, 1H), 6.44 (s, 1H), 6.41 (s, 1H), 7.25-7.43 (m, 4H), 7.63 (d, 1H), 8.01 (q, 1H), 8.18-8.28 (m, 1H).

MS m/z 532 [M+H]⁺

The two enantiomers were arbitrarily assigned stereochemistry:

### Example 9

(R)-6-amino-5-[(4,4-difluoro-1-{[4-(trifluoromethoxy)phenyl]acetyl}pyrrolidin-3-yl)oxy]-N-(2-hydroxy-2-methylpropyl)pyridine-3-carboxamide

### Example 10

(S)-6-amino-5-[(4,4-difluoro-1-{[4-(trifluoromethoxy)phenyl]acetyl}pyrrolidin-3-yl)oxy]-N-(2-hydroxy-2-methyl propyl)pyridine-3-carboxamide

### Examples 11 and 12 (S) and (R)-6-amino-5-[(4,4-difluoro-1-{[4-(trifluoromethoxy)phenyl]acetyl}pyrrolidin-3-yl)methoxy]-N-methylpyridine-3-carboxamide

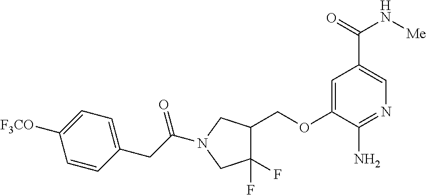 [SMILES: CNC(=O)c1cnc(N)c(OCC2CN(C(=O)Cc3ccc(OC(F)(F)F)cc3)CC2(F)F)c1]

The racemic title compound was prepared according to the method described for Example 1 using racemic-6-amino-5-[(4,4-difluoro-1-{[4-(trifluoromethoxy)phenyl]acetyl}pyrrolidin-3-yl)methoxy]pyridine-3-carboxylic acid (Preparation 5) and methylamine.

The residue was separated into the two enantiomers using chiral column chromatography according to the conditions described below:

Column: Chiralpak AS-5 4.6×100 mm, 5 micron

Mobile phase: 10% MeOH with DEA in water

Flow rate: 4 mL/min.

Rt=2.78 minutes and 3.36 minutes

¹H NMR (400 MHz, DMSO-d₆): δ ppm 2.71-2.78 (m, 3H), 3.41 (dd, 1H), 3.67-3.81 (m, 3H), 3.82-4.02 (m, 2H), 4.08-4.17 (m, 1H), 4.17-4.35 (m, 2H), 6.23 (d, 2H), 7.25-7.33 (m, 2H), 7.33-7.39 (m, 2H), 7.45 (d, 1H), 8.06-8.20 (m, 2H).

The two enantiomers were arbitrarily assigned stereochemistry:

### Example 11

(R)-6-amino-5-[(4,4-difluoro-1-{[4-(trifluoromethoxy)phenyl]acetyl}pyrrolidin-3-yl)methoxy]-N-methylpyridine-3-carboxamide

### Example 12

(S)-6-amino-5-[(4,4-difluoro-1-{[4-(trifluoromethoxy)phenyl]acetyl}pyrrolidin-3-yl)methoxy]-N-methylpyridine-3-carboxamide

### Example 13

### Racemic-6-amino-5-[(4,4-difluoro-1-{[4-(trifluoromethoxy)phenyl]acetyl}pyrrolidin-3-yl)methoxy]pyridine-3-carboxamide

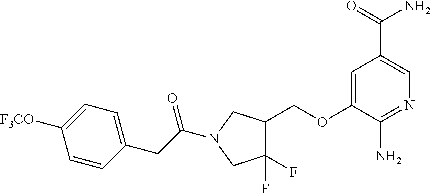 [SMILES: NC(=O)c1cnc(N)c(OCC2CN(C(=O)Cc3ccc(OC(F)(F)F)cc3)CC2(F)F)c1]

The title compound was prepared according to the method described for Example fusing racemic-6-amino-5-[(4,4-difluoro-1-{[4-(trifluoromethoxy)phenyl]acetyl}pyrrolidin-3-yl)methoxy]pyridine-3-carboxylic acid (Preparation 5) and ammonium chloride. The residue was purified using silica gel column chromatography eluting with 15% [7N NH₃ in MeOH] in DCM.

LCMS (Cosmosil 3-HOP; 150×4.6 mm; 4.5 mL/min; 5-50% MeOH).

Rt=2.321 minutes MS m/z 475 [M+H]⁺

### Examples 14 and 15

### (S) and (R)-6-amino-5-[(4,4-difluoro-1-{[4-(trifluoromethoxy)phenyl]acetyl}pyrrolidin-3-yl)methoxy]pyridine-3-carboxamide

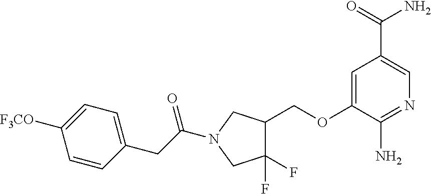 [SMILES: NC(=O)c1cnc(N)c(OCC2CN(C(=O)Cc3ccc(OC(F)(F)F)cc3)CC2(F)F)c1]

Racemic-6-amino-5-[(4,4-difluoro-1-{[4-(trifluoromethoxy)phenyl]acetyl}pyrrolidin-3-yl)methoxy]pyridine-3-carboxamide (Example 13) was separated into the two enantiomers using chiral column chromatography according to the conditions described below:

Column: Lux-Cellulose-1 4.6×100 mm, 3 micron

Mobile phase: 5% MeOH in water

Flow rate: 3 mL/min.

Rt=2.321 minutes and 2.920 minutes

The two enantiomers were arbitrarily assigned stereochemistry:

### Example 14

(S)-6-amino-5-[(4,4-difluoro-1-{[4-(trifluoromethoxy)phenyl]acetyl}pyrrolidin-3-yl)methoxy]pyridine-3-carboxamide

### Example 15

(R)-6-amino-5-[(4,4-difluoro-1-{[4-(trifluoromethoxy)phenyl]acetyl}pyrrolidin-3-yl)methoxy]pyridine-3-carboxamide

### Example 16

### 6-amino-N-methyl-5-[(5-{[4-(trifluoromethoxy)phenyl]acetyl}-5-azaspiro[2.4]hept-7-yl)oxy]pyridine-3-carboxamide

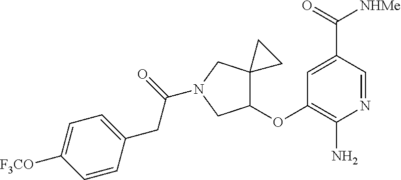 [SMILES: CNC(=O)c1cnc(N)c(OC2CN(C(=O)Cc3ccc(OC(F)(F)F)cc3)CC23CC3)c1]

### Method 1

To a solution of 6-amino-5-(5-azaspiro[2.4]hept-7-yloxy)-N-methylpyridine-3-carboxamide hydrochloride (Preparation 15, 50 mg, 0.191 mmol) in DCM (4 mL) was added triethylamine (0.186 mL, 1.34 mmol), EDCI (55 mg, 0.286 mmol), HOBt (39 mg, 0.286 mmol) followed by 4-trifluoromethoxyphenylacetic acid (38 mg, 0.172 mmol) and the reaction was stirred at room temperature for 16 hours. The reaction was diluted with DCM and washed with saturated aqueous sodium bicarbonate solution, water, brine, dried over sodium sulphate and concentrated in vacuo. The residue was purified using preparative TLC eluting with 5% MeOH in DCM to afford the title compound as a white solid (18 mg, 20%).

¹H NMR (400 MHz, DMSO-d₆): δ ppm 0.62-0.67 (m, 1H), 0.79-0.89 (m, 3H), 2.73-2.74 (m, 3H), 3.36-3.38 (m, 1H), 3.61-3.68 (m, 3H), 3.75-3.87 (m, 1H), 3.97-4.14 (m, 1H), 4.43-4.53 (m, 1H), 6.20-6.23 (m, 2H), 7.22-7.35 (m, 4H), 8.08-8.12 (m, 2H).

MS m/z 465 [M+H]⁺

### Example 17

### (3R,4S)-6-amino-N-methyl-5-[(4-methyl-1-{[4-(trifluoromethoxy)phenyl]acetyl}pyrrolidin-3-yl)oxy]pyridine-3-carboxamide

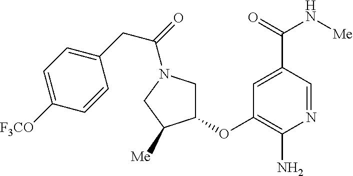 [SMILES: CNC(=O)c1cnc(N)c(O[C@H]2CN(C(=O)Cc3ccc(OC(F)(F)F)cc3)C[C@@H]2C)c1]

### Method 2

A mixture of (3R,4S)-6-amino-5-[(4-methyl-1-{[4-(trifluoromethoxy)phenyl]acetyl}pyrrolidin-3-yl)oxy]pyridine-3-carboxylic acid (Preparation 2, 35 mg, 0.08 mmol), methylamine (2M solution in THF, 0.5 mL) and HATU (45 mg, 0.12 mmol) in pyridine (1 mL) was stirred at room temperature for 20 hours. The reaction was diluted with EtOAc and washed with water, brine, dried over sodium sulphate and concentrated in vacuo. The residue was purified using preparative TLC eluting with 7% MeOH in DCM to afford the title compound as a white solid (15 mg, 42%).

¹H NMR (400 MHz, DMSO-d₆): δ ppm 1.02-1.05 (m, 3H), 2.73-2.75 (m, 3H), 3.20-3.45 (m, 2H), 3.61-3.79 (m, 4H), 3.90-3.99 (m, 1H), 4.61-4.71 (m, 1H), 6.28-6.30 (m, 2H), 7.25-7.35 (m, 4H), 8.09-8.32 (m, 2H).

MS m/z 453 [M+H]⁺

Examples 18-25 were prepared according to Method 1 or Method 2 as described for Examples 16 and 17. The Examples were purified as above or according to one of the methods below:

Purification Method A: Preparative TLC eluting with 7.5% MeOH in DCM.

Purification Method B: Preparative TLC eluting with 10% MeOH in EtOAc.

Purification Method C: Preparative TLC eluting with 5% IPA in DCM.

| Exam- | | | |
|---|---|---|---|
| ple | | | |
| Number | Name/Structure | SM | Data |
| 18 | 6-amino-5-[(5-{[4-(trifluoromethoxy)phenyl]acetyl}- 5-azaspiro[2.4]hept-7-yl)oxy]pyridine-3-carboxamide 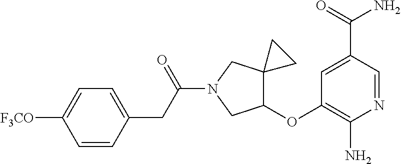 [SMILES: NC(=O)c1cnc(N)c(OC2CN(C(=O)Cc3ccc(OC(F)(F)F)cc3)CC23CC3)c1] | 6-amino-5-(5- azaspiro[2.4]hept- 7-yloxy)pyridine- 3-carboxamide (Preparation 16) and 4-trifluoro- methoxyphenyl- acetic acid. | ¹H NMR (400 MHz, DMSO-d₆): δ ppm 0.62-0.65 (m, 1H), 0.79-0.89 (m, 3H), 3.19-3.36 (m, 1H), 3.64-3.87 (m, 4H), 4.00-4.13 (m, 1H), 4.44-4.53 (m, 1H), 6.21-6.23 (br m, 2H), 7.06 (br s, 1H), 7.22- 7.38 (m, 5H), 7.70 (br s, 1H), 8.14 (s, 1H). MS m/z 451 [M + H]⁺ |
| | | | PM A. |
| 19 | (R)-6-amino-5-[(4,4-dimethyl-1-{[4-(trifluoromethoxy) phenyl]acetyl}pyrrolidin-3-yl)oxy]pyridine-3- carboxamide 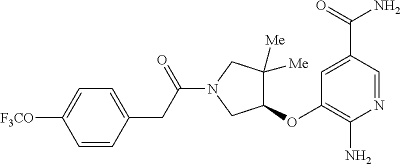 [SMILES: CC1(C)CN(C(=O)Cc2ccc(OC(F)(F)F)cc2)C[C@@H]1Oc1cc(C(N)=O)cnc1N] | (R)-6-amino-5- [(4,4- dimethylpyrrolidin- 3-yl)oxy]pyridine- 3-carboxamide hydrochloride (Preparation 17) and 4- trifluoromethoxy- phenylacetic acid. | ¹H NMR (400 MHz, DMSO-d₆): δ ppm 1.06 (s, 3H), 1.17 (s, 3H), 3.35-3.75 (m, 5H), 3.80-4.05 (m, 1H), 4.50-4.60 (m, 1H), 6.27-6.30 (m, 2H), 7.08 (br s, 1H), 7.23-7.39 (m, 5H), 7.68 (br s, 1H), 8.13 (s, 1H). MS m/z 453 [M + H]⁺ PM B. |
| 20 | (R)-6-amino-5-[(4,4-dimethyl-1-{[4-(trifluoromethoxy) phenyl]acetyl}pyrrolidin-3-yl)oxy]-N- methylpyridine-3-carboxamide 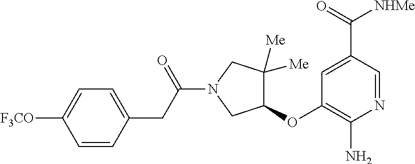 [SMILES: CNC(=O)c1cnc(N)c(O[C@H]2CN(C(=O)Cc3ccc(OC(F)(F)F)cc3)CC2(C)C)c1] | (R)-6-amino-5- [(4,4- dimethylpyrrolidin- 3-yl)oxy]-N- methylpyridine-3- carboxamide hydrochloride (Preparation 18) and 4-trifluoro- methoxyphenyl- acetic acid. | ¹H NMR (400 MHz, DMSO-d₆): δ ppm 1.08 (s, 3H), 1.17 (s, 3H), 2.74 (s, 3H), 3.31-3.70 (m, 5H), 4.50-4.60 (m, 1H), 6.25-6.27 (m, 2H), 7.25-7.36 (m, 5H), 8.08-8.10 (m, 2H). MS m/z 467 [M + H]⁺ PM C. |
| 21 | (3R,4S)-6-amino-N-ethyl-5-[(4-methyl-1-{[4-(trifluoromethoxy) phenyl]acetyl}pyrrolidin-3-yl)oxy]pyridine-3-carboxamide 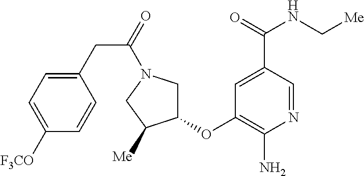 [SMILES: CCNC(=O)c1cnc(N)c(O[C@H]2CN(C(=O)Cc3ccc(OC(F)(F)F)cc3)C[C@@H]2C)c1] | (3R,4S)-6- amino-5-[(4- methyl-1-{[4- (trifluoromethoxy) phenyl]acetyl} pyrrolidin-3- yl)oxy]pyridine-3- carboxylic acid (Preparation 2) and ethylamine. | ¹H NMR (400 MHz, DMSO-d₆): δ ppm 1.02-1.12 (m, 6H), 2.49-2.60 (m, 1H), 3.20-3.45 (m, 4H), 3.64-3.75 (m, 3H), 3.90-4.05 (m, 1H), 4.62-4.72 (m, 1H), 6.27-6.29 (m, 2H), 7.24-7.36 (m, 5H), 8.11-8.17 (m, 2H). MS m/z 467 [M + H]⁺ |
| 22 | (3R,4S)-6-amino-N-(cyanomethyl)-5-[(4-methyl-1-{[4- (trifluoromethoxy)phenyl]acetyl}pyrrolidin-3- yl)oxy]pyridine-3-carboxamide 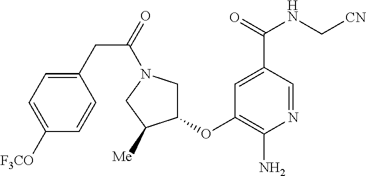 [SMILES: C[C@H]1CN(C(=O)Cc2ccc(OC(F)(F)F)cc2)C[C@@H]1Oc1cc(C(=O)NCC#N)cnc1N] | (3R,4S)-6- amino-5-[(4- methyl-1-{[4- (trifluoromethoxy) phenyl]acetyl} pyrrolidin-3- yl)oxy]pyridine-3- carboxylic acid (Preparation 2) and aminoacetonitrile. | ¹H NMR (400 MHz, DMSO-d₆): δ ppm 1.02-1.23 (m, 3H), 2.40-2.60 (m, 1H), 3.20-3.46 (m, 2H), 3.62-3.79 (m, 3H), 3.89-4.02 (m, 1H), 4.27 (s, 2H), 4.63- 4.73 (m, 1H), 6.48- 6.50 (m, 2H), 7.24- 7.37 (m, 5H), 8.13 (s, 1H), 8.84-8.86 (m, 1H). MS m/z 478 [M + H]⁺ |
| 23 | (3R,4S)-6-amino-N-(2-hydroxy-2-methylpropyl)-5- [(4-methyl-1-{[4-(trifluoromethoxy)phenyl]acetyl} pyrrolidin-3-yl)oxy]pyridine-3-carboxamide 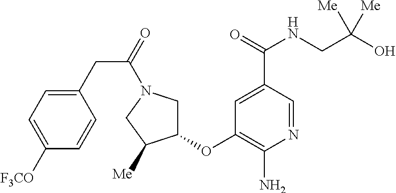 [SMILES: C[C@H]1CN(C(=O)Cc2ccc(OC(F)(F)F)cc2)C[C@@H]1Oc1cc(C(=O)NCC(C)(C)O)cnc1N] | (3R,4S)-6- amino-5-[(4- methyl-1-{[4- (trifluoromethoxy) phenyl]acetyl} pyrrolidin-3- yl)oxy]pyridine-3- carboxylic acid (Preparation 2) and 1-amino-2- methylpropan-2-ol. | ¹H NMR (400 MHz, DMSO-d₆): δ ppm 1.02-1.08 (m, 9H), 3.20-3.45 (m, 4H), 3.60-3.75 (m, 4H), 3.80-4.05 (m, 1H), 4.55 (s, 1H), 4.65- 4.70 (m, 1H), 6.30- 6.32 (m, 2H), 7.24- 7.40 (m, 5H), 8.02- 8.04 (m, 1H), 8.15 (s, 1H). MS m/z 511 [M + H]⁺ |
| 24 | Racemic-6-amino-5-[(4-methoxy-4-methyl-1-{[4- (trifluoromethoxy)phenyl]acetyl}pyrrolidin-3-yl)oxy]-N- methylpyridine-3-carboxamide 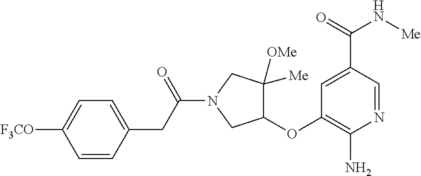 [SMILES: CNC(=O)c1cnc(N)c(OC2CN(C(=O)Cc3ccc(OC(F)(F)F)cc3)CC2(C)OC)c1] | Racemic-6- amino-5-[(4- methoxy-4- methyl-1-{[4- (trifluoromethoxy) phenyl]acetyl} pyrrolidin-3- yl)oxy]pyridine-3- carboxylic acid (Preparation 6) and methylamine. | LCMS (Zymor Diol/Monol; 5-50% MeOH @ 15%/min; 5.6 mL/min): Rt = 1.37 minutes MS m/z 483 [M + H]⁺ |
| 25 | Racemic-6-amino-N,N-diethyl-5-[(4-methoxy-4- methyl-1-{[4-(trifluoromethoxy)phenyl]acetyl} pyrrolidin-3-yl)oxy]pyridine-3-carboxamide 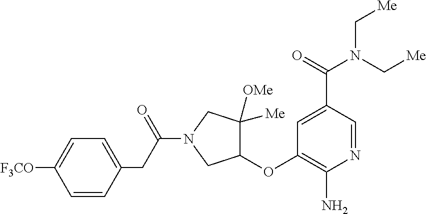 [SMILES: CCN(CC)C(=O)c1cnc(N)c(OC2CN(C(=O)Cc3ccc(OC(F)(F)F)cc3)CC2(C)OC)c1] | Racemic-6- amino-5-[(4- methoxy-4- methyl-1-{[4- (trifluoromethoxy) phenyl]acetyl} pyrrolidin-3- yl)oxy]pyridine-3- carboxylic acid (Preparation 6) and diethylamine. | LCMS (Zymor Diol/Monol; 5-50% MeOH @ 15%/min; 5.6 mL/min): Rt = 1.08 minutes MS m/z 525 [M + H]⁺ |

### Preparation 1

### (S)-6-amino-5-{(4,4-difluoro-1-{[4-(trifluoromethoxy)phenyl]acetyl}pyrrolidin-3-yl)oxy}pyridine-3-carboxylic acid

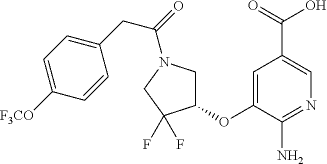 [SMILES: Nc1ncc(C(=O)O)cc1O[C@H]1CN(C(=O)Cc2ccc(OC(F)(F)F)cc2)CC1(F)F]

To a solution of (S)-methyl-6-amino-5-[(4,4-difluoro-1-{[4-(trifluoromethoxy)phenyl]acetyl}pyrrolidin-3-yl)oxy]pyridine-3-carboxylate (Preparation 7, 438 mg, 0.92 mmol) in MeOH (6 mL) was added 2N aqueous sodium hydroxide (2.3 mL) and the reaction was heated to 60° C. for 5 minutes. The reaction was cooled, acidified with cHCl (aq) and concentrated in vacuo, azeotroping with MeCN to afford the title compound that was taken on directly to the next step.

### Preparation 2

### (3R,4S)-6-amino-5-[(4-methyl-1-{[4-(trifluoromethoxy)phenyl]acetyl}pyrrolidin-3-yl)oxy]pyridine-3-carboxylic acid

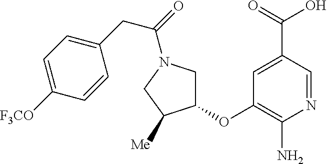 [SMILES: C[C@H]1CN(C(=O)Cc2ccc(OC(F)(F)F)cc2)C[C@@H]1Oc1cc(C(=O)O)cnc1N]

To a solution of (3R,4S)-methyl 6-amino-5-[(4-methyl-1-{[4-(trifluoromethoxy)phenyl]acetyl}pyrrolidin-3-yl)oxy]pyridine-3-carboxylate (Preparation 10, 140 mg, 0.309 mmol) in THF (0.75 mL) and water (0.5 mL) was added lithium hydroxide (25 mg, 0.618 mmol) and the reaction was stirred at room temperature for 14 hours. The reaction was diluted with water and washed with diethyl ether. The aqueous layer was collected and acidified to pH=6 with 6N HCl (aq). The resulting suspension was filtered and washed with diethyl ether to afford the title compound as a brown solid (70 mg, 52%).

MS m/z 440 [M+H]⁺

### Preparation 3

### Racemic-6-amino-5-[(4,4-difluoro-1-{[4-(trifluoromethoxy)phenyl]acetyl}pyrrolidin-3-yl)oxy]pyridine-3-carboxylic acid

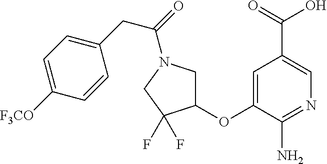 [SMILES: Nc1ncc(C(=O)O)cc1OC1CN(C(=O)Cc2ccc(OC(F)(F)F)cc2)CC1(F)F]

The title compound was prepared according to the methods described for the total synthesis of (S)-6-amino-5-[(4,4-difluoro-1-{[4-(trifluoromethoxy)phenyl]acetyl}pyrrolidin-3-yl)oxy]pyridine-3-carboxylic acid (Preparation 1) using racemic-tert-butyl-3-((tert-butyldimethylsilyl)oxy)-4-oxopyrrolidine-1-carboxylate (WO2014075392).

Preparations 4-6 were prepared according to either Preparation 1 or 2 as described above.

| Prep | | | |
|---|---|---|---|
| Num- | | | |
| ber | Name/Structure | SM | Data |
| 4 | Racemic-6-amino-5-{[(4,4-difluoro-1-{[4- (trifluoromethoxy)phenyl]acetyl}pyrrolidin-3- yl)oxy]methyl}pyridine-3-carboxylic acid 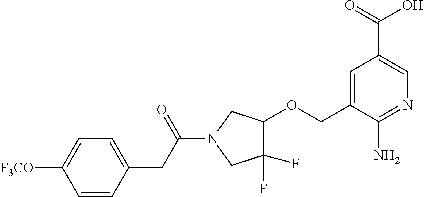 [SMILES: Nc1ncc(C(=O)O)cc1COC1CN(C(=O)Cc2ccc(OC(F)(F)F)cc2)CC1(F)F] | Racemic-methyl- 6-amino-5-{[(4,4- difluoro-1-{[4- (trifluoromethoxy) phenyl]acetyl} pyrrolidin-3- yl)oxy]methyl} pyridine-3- carboxylate (Preparation 8). | Taken on directly to the next step. |
| 5 | Racemic-6-amino-5-[(4,4-difluoro-1-{[4- (trifluoromethoxy)phenyl]acetyl}pyrrolidin-3- yl)methoxy]pyridine-3-carboxylic acid 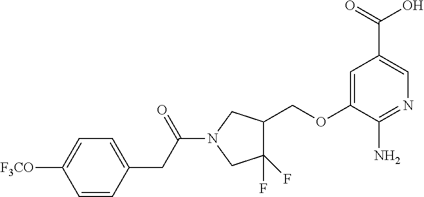 [SMILES: Nc1ncc(C(=O)O)cc1OCC1CN(C(=O)Cc2ccc(OC(F)(F)F)cc2)CC1(F)F] | Racemic-methyl- 6-amino-5-[(4,4- difluoro-1-{[4- (trifluoromethoxy) phenyl]acetyl} pyrrolidin-3- yl)methoxy] pyridine-3-carbox- ylate (Preparation 9). | Taken on directly to the next step. |
| 6 | Racemic-6-amino-5-[(4-methoxy-4-methyl-1-{[4- (trifluoromethoxy)phenyl]acetyl}pyrrolidin-3- yl)oxy]pyridine-3-carboxylic acid 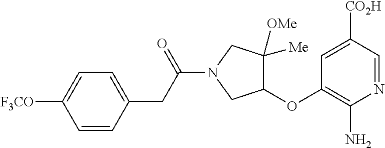 [SMILES: COC1(C)CN(C(=O)Cc2ccc(OC(F)(F)F)cc2)CC1Oc1cc(C(=O)O)cnc1N] | Racemic-methyl- 6-amino-5-[(4- methoxy-4- methyl-1-{[4- (trifluoromethoxy) phenyl]acetyl} pyrrolidin-3- yl)oxy]pyridine-3- carboxylate (Preparation 11). | Taken on directly to the next step. |

### Preparation 7

### (S)-Methyl 6-amino-5-[(4,4-difluoro-1-{[4-(trifluoromethoxy)phenyl]acetyl}pyrrolidin-3-yl)oxy]pyridine-3-carboxylate

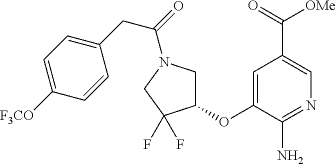 [SMILES: COC(=O)c1cnc(N)c(O[C@H]2CN(C(=O)Cc3ccc(OC(F)(F)F)cc3)CC2(F)F)c1]

To a solution of (S)-methyl 6-amino-5-[(4,4-difluoropyrrolidin-3-yl)oxy]pyridine-3-carboxylate hydrochloride (Preparation 12, 1.30 g, 4.77 mmol) in DMF (20 mL) was added 2-(4-(trifluoromethoxy)phenyl)acetic acid (1.05 g, 4.77 mmol) followed by triethylamine (2.66 mL, 19.1 mmol) and HATU (2.72 g, 7.16 mmol). The reaction was stirred at room temperature for 30 minutes before being diluted with water and extracted into EtOAc (2×60 mL). The organic layers were combined, washed with 10% aqueous potassium carbonate solution (2×60 mL), water (60 mL), brine (60 mL), dried over sodium sulphate and concentrated in vacuo. The residue was purified using silica gel column chromatography eluting with 20-100% EtOAc in heptanes to afford the title compound as a yellow foam (2.23 g, 98%).

¹H NMR (400 MHz, DMSO-d₆): δ ppm 3.64-3.84 (m, 5H), 3.88-3.98 (m, 1H), 4.05-4.15 (m, 1H), 4.20-4.44 (m, 1H), 5.35-5.48 (m, 2H), 6.75-6.90 (m, 2H), 7.25-7.38 (m, 4H), 7.54-7.60 (m, 1H), 8.25 (m, 1H).

MS m/z 476 [M+H]⁺

### Preparation 8

### Racemic-methyl 6-amino-5-{[(4,4-difluoro-1-{[4-(trifluoromethoxy)phenyl]acetyl}pyrrolidin-3-yl)oxy]methyl}pyridine-3-carboxylate

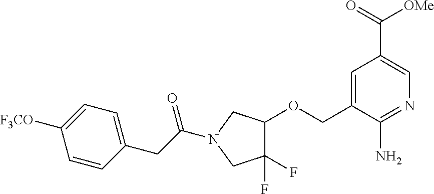 [SMILES: COC(=O)c1cnc(N)c(COC2CN(C(=O)Cc3ccc(OC(F)(F)F)cc3)CC2(F)F)c1]

The title compound was prepared according to the method described for Preparation 7 using racemic-methyl 6-amino-5-{[(4,4-difluoropyrrolidin-3-yl)oxy]methyl}pyridine-3-carboxylate trifluoroacetate (Preparation 13).

¹H NMR (400 MHz, CDCl₃): δ ppm 3.63 (s, 2H), 3.74-3.97 (m, 7H), 4.11 (s, 1H), 4.59-4.82 (m, 2H), 6.07 (br s, 1H), 6.20 (br s, 1H), 7.15-7.23 (m, 2H), 7.29 (m, 2H), 8.04-8.06 (m, 1H), 8.57-8.73 (m, 1H).

### Preparation 9

### Racemic-methyl-6-amino-5-[(4,4-difluoro-1-{[4-(trifluoromethoxy)phenyl]acetyl}pyrrolidin-3-yl)methoxy]pyridine-3-carboxylate

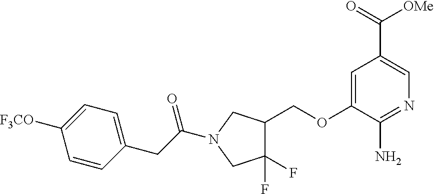 [SMILES: COC(=O)c1cnc(N)c(OCC2CN(C(=O)Cc3ccc(OC(F)(F)F)cc3)CC2(F)F)c1]

The title compound was prepared according to the method described for Preparation 7 using racemic-methyl 6-amino-5-[(4,4-difluoropyrrolidin-3-yl)methoxy]pyridine-3-carboxylate (Preparation 14) in DMA.

LCMS (XBridge C18 2.1×30 mm, 2.5 micron, 5-95% MeOH in 2.5 minutes).

Rt=1.70 minutes MS m/z 490 [M+H]⁺

### Preparation 10

### (3R,4S)-Methyl 6-amino-5-[(4-methyl-1-{[4-(trifluoromethoxy)phenyl]acetyl}pyrrolidin-3-yl)oxy]pyridine-3-carboxylate

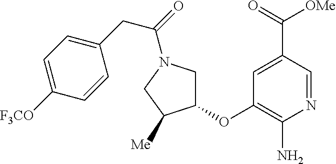 [SMILES: COC(=O)c1cnc(N)c(O[C@H]2CN(C(=O)Cc3ccc(OC(F)(F)F)cc3)C[C@@H]2C)c1]

The title compound was prepared according to the method described by Example 16 using (3R,4S)-methyl-6-amino-5-[(4-methylpyrrolidin-3-yl)oxy]pyridine-3-carboxylate hydrochloride (Preparation 19) and 4-trifluoromethoxyphenylacetic acid. The residue was purified using silica gel column chromatography eluting with 0-2% MeOH in DCM.

¹H NMR (400 MHz, DMSO-d₆): δ ppm 1.02 (m, 3H), 3.32-3.45 (m, 1H), 3.61-3.82 (m, 8H), 3.80-4.00 (m, 1H), 4.70-4.85 (m, 1H), 6.71 (br s, 2H), 7.24-7.36 (m, 5H), 8.19 (s, 1H).

MS m/z 454 [M+H]⁺

### Preparation 11

### Racemic-methyl 6-amino-5-[(4-methoxy-4-methyl-1-{[4-(trifluoromethoxy)phenyl]acetyl}pyrrolidin-3-yl)oxy]pyridine-3-carboxylate

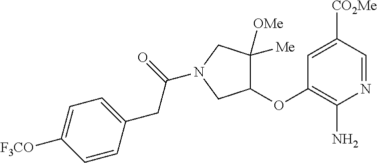 [SMILES: COC(=O)c1cnc(N)c(OC2CN(C(=O)Cc3ccc(OC(F)(F)F)cc3)CC2(C)OC)c1]

The title compound was prepared according to the method described by Example 16 using racemic-methyl 6-amino-5-[(4-methoxy-4-methylpyrrolidin-3-yl)oxy]pyridine-3-carboxylate (Preparation 20).

¹H NMR (400 MHz, DMSO-d₆): δ ppm 1.40-1.45 (m, 3H), 3.30 (s, 3H), 3.60-4.00 (m, 9H), 4.80-4.90 (m, 1H), 6.35 (br s, 2H), 7.10-7.50 (m, 5H), 8.20 (s, 1H).

### Preparation 12

### (S)-methyl 6-amino-5-[(4,4-difluoropyrrolidin-3-yl)oxy]pyridine-3-carboxylate hydrochloride

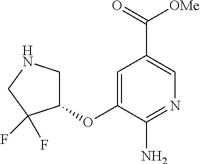 [SMILES: COC(=O)c1cnc(N)c(O[C@H]2CNCC2(F)F)c1]

A solution of (S)-methyl 6-amino-5-{[1-(tert-butoxycarbonyl)-4,4-difluoropyrrolidin-3-yl]oxy}pyridine-3-carboxylate (Preparation 25, 1.78 g, 4.77 mmol) in 4N HCl in dioxane (22 mL, 88 mmol) was stirred at room temperature for 90 minutes. The reaction was concentrated in vacuo and azeotroped with DCM to afford the title compound that was used directly in the next step.

### Preparation 13

### Racemic-methyl 6-amino-5-{[(4,4-difluoropyrrolidin-3-yl)oxy]methyl}pyridine-3-carboxylate trifluoroacetate

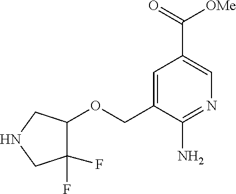 [SMILES: COC(=O)c1cnc(N)c(COC2CNCC2(F)F)c1]

A solution of racemic-methyl 6-amino-5-({[1-(3,3-dimethylbutanoyl)-4,4-difluoropyrrolidin-3-yl]oxy}methyl)pyridine-3-carboxylate (Preparation 27, 4.46 mg, 1.15 mmol) in TFA (2 mL) was stirred at room temperature for 10 minutes. The reaction was concentrated in vacuo and used directly in the next reaction as the title compound.

Preparations 14-20 were prepared according to either Preparation 12 or 13 as described above. The associated salts are included in the name.

| Prep | | | |
|---|---|---|---|
| Number | Name/Structure | SM | Data |
| 14 | Racemic-methyl 6-amino-5-[(4,4- difluoropyrrolidin-3-yl)methoxy] pyridine-3-carboxylate trifluoro- acetate 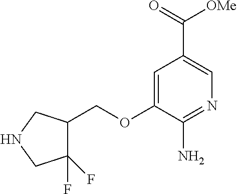 [SMILES: COC(=O)c1cnc(N)c(OCC2CNCC2(F)F)c1] | Racemic-methyl 6- amino-5-{[1-(tert- butoxycarbonyl)-4,4- difluoropyrrolidin-3- yl]methoxy}pyridine- 3-carboxylate (Preparation 28) | Taken on directly to the next step. |
| 15 | Racemic-6-amino-5-(5-azaspiro[2.4] hept-7-yloxy)-N-methylpyridine-3- carboxamide hydrochloride 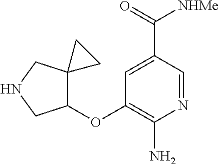 [SMILES: CNC(=O)c1cnc(N)c(OC2CNCC23CC3)c1] | tert-butyl 7-{[2- amino-5-(methoxy carbonyl)pyridin- 3-yl]oxy}-5- azaspiro[2.4]heptane- 5-carboxylate (Preparation 21). | MS m/z 263 [M + H]⁺ |
| 16 | Racemic-6-amino-5-(5-azaspiro[2.4] hept-7-yloxy)pyridine-3- carboxamide hydrochloride 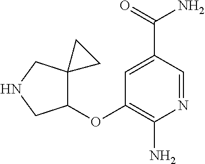 [SMILES: NC(=O)c1cnc(N)c(OC2CNCC23CC3)c1] | Racemic-tert-butyl 7-[(2-amino-5- carbamoylpyridin-3- yl)oxy]-5-azaspiro[2.4] heptane-5-carboxylate (Preparation 23). | MS m/z 249 [M + H]⁺ |
| 17 | (R)-6-amino-5-[(4,4-dimethyl- pyrrolidin-3-yl)oxy]pyridine- 3-carboxamide hydrochloride 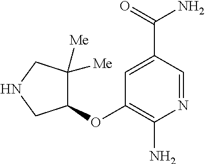 [SMILES: CC1(C)CNC[C@@H]1Oc1cc(C(N)=O)cnc1N] | (R)-tert-butyl 4-[(2- amino-5- carbamoylpyridin-3- yl)oxy]-3,3- dimethylpyrrolidine- 1-carboxylate (Preparation 24). | Taken on directly to the next step. |
| 18 | (R)-6-amino-5-[(4,4-dimethyl- pyrrolidin-3-yl)oxy]-N-methyl- pyridine-3-carboxamide hydrochloride 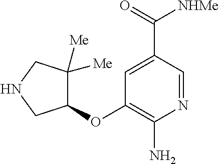 [SMILES: CNC(=O)c1cnc(N)c(O[C@H]2CNCC2(C)C)c1] | (R)-tert-butyl 4-{[2- amino-5-(methyl- carbamoyl)pyridin- 3-yl]oxy}-3,3- dimethylpyrrolidine- 1-carboxylate (Preparation 22). | MS m/z 265 [M + H]⁺ |
| 19 | (3R,4S)-Methyl 6-amino-5- [(4-methylpyrrolidin-3- yl)oxy]pyridine-3-carboxylate hydrochloride 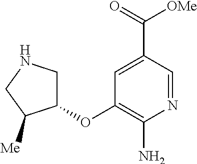 [SMILES: COC(=O)c1cnc(N)c(O[C@H]2CNC[C@@H]2C)c1] | (3R,4S)-methyl 6- amino-5-{[1-(tert- butoxycarbonyl)-4- methylpyrrolidin-3- yl]oxy}pyridine-3- carboxylate (Preparation 31). | MS m/z 251 [M + H]⁺ |
| 20 | Racemic-methyl 6-amino-5- [(4-methoxy-4-methyl pyrrolidin-3-yl)oxy]pyridine- 3-carboxylate hydrochloride 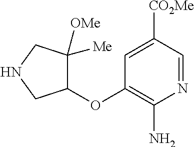 [SMILES: COC(=O)c1cnc(N)c(OC2CNCC2(C)OC)c1] | Racemic-tert-butyl 4-[(2-amino-5- bromopyridin-3- yl)oxy]-3-methoxy-3- methylpyrrolidine-1- carboxylate (Preparation 20). | Taken on directly to the next step. |

### Preparation 21 tert-butyl 7-{[2-amino-5-(methylcarbamoyl)pyridin-3-yl]oxy}-5-azaspiro[2.4]heptane-5-carboxylate

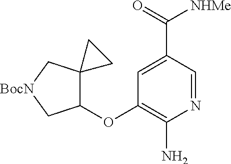 [SMILES: CNC(=O)c1cnc(N)c(OC2CN(C(=O)OC(C)(C)C)CC23CC3)c1]

A solution of tert-butyl 7-{[2-amino-5-(methoxycarbonyl)pyridin-3-yl]oxy}-5-azaspiro[2.4]heptane-5-carboxylate (Preparation 26, 100 mg, 0.26 mmol) in 1M methylamine in MeOH (10 mL) was heated at 50° C. in a sealed tube for 60 hours. The reaction was concentrated in vacuo and purified using silica gel column chromatography eluting with 2% MeOH in DCM to afford the title compound as a yellow gum (70 mg, 74%).

MS m/z 363 [M+H]⁺

### Preparation 22

### (R)-tert-butyl 4-{[2-amino-5-(methylcarbamoyl)pyridin-3-yl]oxy}-3,3-dimethylpyrrolidine-1-carboxylate

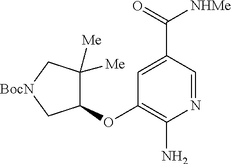 [SMILES: CNC(=O)c1cnc(N)c(O[C@H]2CN(C(=O)OC(C)(C)C)CC2(C)C)c1]

The title compound was prepared according to the method described for Preparation using (R)-methyl 6-amino-5-{[1-(tert-butoxycarbonyl)-4,4-dimethylpyrrolidin-3-yl]oxy}pyridine-3-carboxylate (Preparation 30).

MS m/z 365 [M+H]⁺

### Preparation 23

### tert-butyl 7-[(2-amino-5-carbamoylpyridin-3-yl)oxy]-5-azaspiro[2.4]heptane-5-carboxylate

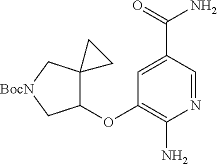 [SMILES: CC(C)(C)OC(=O)N1CC(Oc2cc(C(N)=O)cnc2N)C2(CC2)C1]

To a solution of racemic-tert-butyl 7-[(2-amino-5-cyanopyridin-3-yl)oxy]-5-azaspiro[2.4]heptane-5-carboxylate (Preparation 32, 200 mg, 0.606 mmol) in MeOH (10 mL) was added lithium hydroxide (51 mg, 1.212 mmol) followed by hydrogen peroxide (30% aqueous, 0.25 mL) at 0° C. The reaction was stirred at room temperature for 3 hours. The reaction was diluted with water and brine, dried over sodium sulphate and concentrated in vacuo. The residue was purified using silica gel column chromatography eluting with 3-4% MeOH in DCM to afford the title compound as a yellow gum (120 mg, 57%).

¹H NMR (400 MHz, DMSO-d₆): δ ppm 0.64-0.68 (m, 1H), 0.70-0.80 (m, 2H), 0.82-0.90 (m, 1H), 1.39 (s, 9H), 3.02-3.06 (m, 1H), 3.40-3.47 (m, 1H), 3.65-3.72 (m, 1H), 3.78-3.83 (m, 1H), 4.38-4.40 (m, 1H), 6.20-6.22 (m, 2H), 7.06 (br s, 1H), 7.33 (d, 1H), 7.68 (br s, 1H), 8.13 (d, 1H).

MS m/z 349 [M+H]⁺

### Preparation 24

### (R)-tert-butyl 4-[(2-amino-5-carbamoylpyridin-3-yl)oxy]-3,3-dimethylpyrrolidine-1-carboxylate

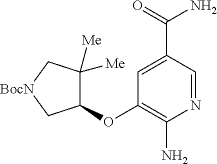 [SMILES: CC(C)(C)OC(=O)N1C[C@H](Oc2cc(C(N)=O)cnc2N)C(C)(C)C1]

The title compound was prepared according to the method described for Preparation 23 using (R)-tert-butyl 4-[(2-amino-5-cyanopyridin-3-yl)oxy]-3,3-dimethylpyrrolidine-1-carboxylate (Preparation 33).

¹H NMR (400 MHz, DMSO-d₆): δ ppm 1.05 (s, 3H), 1.14 (s, 3H), 1.39 (s, 9H), 3.12-3.22 (m, 2H), 3.39-3.43 (m, 1H), 3.72-3.75 (m, 1H), 4.44-4.49 (m, 1H), 6.26-6.28 (br m, 2H), 7.08 (br s, 1H), 7.36 (d, 1H), 7.70 (br s, 1H), 8.13 (d, 1H).

MS m/z 351 [M+H]⁺

### Preparation 25

### (S)-methyl 6-amino-5-{[1-(tert-butoxycarbonyl)-4,4-difluoropyrrolidin-3-yl]oxy}Pyridine-3-carboxylate

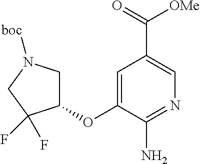

To a solution of (S)-tert-butyl 4-[(2-amino-5-bromopyridin-3-yl)oxy]-3,3-difluoropyrrolidine-1-carboxylate (Preparation 34, 700 mg, 1.78 mmol) in MeOH (15 mL) was added Pd(dppf)Cl₂ (140 mg, 0.18 mmol) and the reaction was heated to 80° C. under an atmosphere of carbon monoxide (80 psi) for 1 hour. Triethylamine (1.11 mL, 7.96 mmol) was added and the reaction was continued at 80° C. under an atmosphere of carbon dioxide (80 psi) for 22 hours. The reaction was cooled and concentrated in vacuo. The residue was purified using silica gel column chromatography eluting with 20-40% EtOAc in heptanes to afford the title compound as a white solid (495 mg, 75%).

¹H NMR (400 MHz, DMSO-d₆): δ ppm 1.40 (s, 9H), 3.50-3.55 (m, 1H), 3.75-3.85 (m, 5H), 3.90-4.05 (m, 1H), 5.25-5.35 (br m, 1H), 6.80 (br s, 2H), 7.55 (s, 1H), 8.20 (s, 1H).

MS m/z 374 [M+H]⁺

### Preparation 26

### tert-butyl 7-{[2-amino-5-(methoxycarbonyl)pyridin-3-yl]oxy}-5-azaspiro[2.4]heptane-5-carboxylate

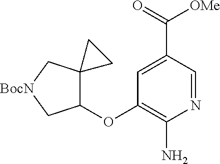 [SMILES: COC(=O)c1cnc(N)c(OC2CN(C(=O)OC(C)(C)C)CC23CC3)c1]

To a solution of tert-butyl 7-[(2-amino-5-bromopyridin-3-yl)oxy]-5-azaspiro[2.4]heptane-5-carboxylate (Preparation 36, 50 mg, 0.13 mmol) in MeOH (2 mL) was added dppp (5.36 mg, 0.013 mmol) and DIPEA (0.6 mL) and the reaction was degassed with carbon monoxide. Pd(OAc)₂ (2.92 mg, 0.013 mmol) was added and the reaction stirred under a balloon of carbon monoxide at 100° C. for 16 hours. The reaction was cooled, diluted with EtOAc and washed five times with water. The organic layer was collected, washed with brine, dried over sodium sulphate and concentrated in vacuo. The residue was purified using silica gel column chromatography eluting with 3-5% MeOH in DCM to afford the title compound as a yellow solid (15 mg, 32%).

MS m/z 364 [M+H]⁺

### Preparation 27

### Racemic-methyl 6-amino-5-({[1-(3,3-dimethylbutanoyl)-4,4-difluoropyrrolidin-3-yl]oxy}methyl)pyridine-3-carboxylate

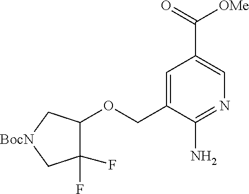 [SMILES: COC(=O)c1cnc(N)c(COC2CN(C(=O)OC(C)(C)C)CC2(F)F)c1]

The title compound was prepared according to the method described for Preparation 25 using racemic-1-{4-[(2-amino-5-bromopyridin-3-yl)methoxy]-3,3-difluoropyrrolidin-1-yl}-3,3-dimethylbutan-1-one (Preparation 39). The residue was purified using silica gel column chromatography eluting with 60% EtOAc in heptanes.

¹H NMR (400 MHz, CDCl₃): δ ppm 1.47 (s, 9H), 3.55 (br s, 1H), 3.65-3.83 (m, 3H), 3.91 (s, 3H), 4.00 (br s, 1H), 4.60-4.70 (m, 1H), 4.76 (br s, 1H), 5.92 (br s, 2H), 8.01 (s, 1H) 8.69 (s, 1H).

### Preparation 28

### Racemic-methyl 6-amino-5-{[1-(tert-butoxycarbonyl)-4,4-difluoropyrrolidin-3-yl]methoxy}pyridine-3-carboxylate

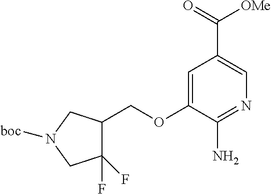

The title compound was prepared according to the method described for Preparation 25 using racemic-tert-butyl 4-{[(2-amino-5-bromopyridin-3-yl)oxy]methyl}-3,3-difluoropyrrolidine-1-carboxylate (Preparation 35). The residue was purified using silica gel column chromatography eluting with 0-50% EtOAc in heptanes.

¹H NMR (400 MHz, CDCl₃): δ ppm 1.40 (s, 9H), 2.90-3.00 (m, 1H), 3.35-3.40 (m, 1H), 3.60-3.85 (m, 6H), 4.00-4.20 (m, 2H), 5.10-5.20 (br s, 2H), 7.40 (s, 1H), 8.30 (s, 1H).

### Preparation 29

### Racemic-methyl 6-amino-5-{[1-(tert-butoxycarbonyl)-4-methoxy-4-methylpyrrolidin-3-yl]oxy}pyridine-3-carboxylate

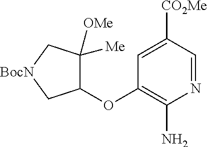 [SMILES: COC(=O)c1cnc(N)c(OC2CN(C(=O)OC(C)(C)C)CC2(C)OC)c1]

The title compound was prepared according to the method described for using racemic-tert-butyl-4-[(2-amino-5-bromopyridin-3-yl)oxy]-3-methoxy-3-methylpyrrolidine-1-carboxylate (Preparation 38) and dppf with triethylamine as base at 80° C. The residue was purified using silica gel column chromatography eluting with 0-100% EtOAc in heptanes.

¹H NMR (400 MHz, CDCl₃): δ ppm 1.40-1.60 (m, 12H), 3.30 (s, 3H), 3.32-3.50 (m, 1H), 3.65-3.90 (m, 6H), 4.60 (s, 1H), 5.00 (br s, 2H), 7.40 (s, 1H), 8.40 (s, 1H).

### Preparation 30

### (R)-methyl-6-amino-5-{[1-(tert-butoxycarbonyl)-4,4-dimethylpyrrolidin-3-yl]oxy}Pyridine-3-carboxylate

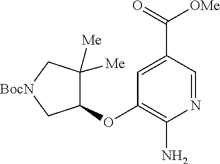 [SMILES: COC(=O)c1cnc(N)c(O[C@H]2CN(C(=O)OC(C)(C)C)CC2(C)C)c1]

The title compound was prepared according to the method described for Preparation 26 using (R)-tert-butyl 4-[(2-amino-5-bromopyridin-3-yl)oxy]-3,3-dimethylpyrrolidine-1-carboxylate (Preparation 37) at 80° C. The residue was purified using silica gel column chromatography eluting with 40-50% EtOAc in heptanes.

MS m/z 366 [M+H]⁺

### Preparation 31

### (3R,4S)-Methyl 6-amino-5-{[1-(tert-butoxycarbonyl)-4-methylpyrrolidin-3-yl]oxy}pyridine-3-carboxylate

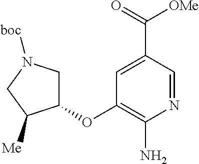

To a solution of methyl 6-amino-5-hydroxypyridine-3-carboxylate (Preparation 57, 380 mg, 2.29 mmol) and (3S,4S)-tert-butyl 3-methyl-4-[(methylsulfonyl)oxy]pyrrolidine-1-carboxylate (Preparation 56, 702 mg, 2.52 mmol) in DMF (5 mL) was added cesium carbonate (2.23 g, 6.87 mmol) and the reaction was heated to 130° C. for 15 hours. The reaction was cooled and diluted with EtOAc, washed with water, brine, dried over sodium sulphate and concentrated in vacuo. The residue was purified using silica gel column chromatography eluting with 40% EtOAc in heptanes to afford the title compound as a brown gum (200 mg, 25%).

MS m/z 352 [M+H]⁺

### Preparation 32

### tert-butyl 7-[(2-amino-5-cyanopyridin-3-yl)oxy]-5-azaspiro[2.4]heptane-5-carboxylate

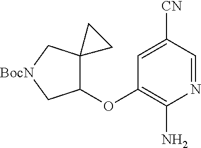 [SMILES: CC(C)(C)OC(=O)N1CC(Oc2cc(C#N)cnc2N)C2(CC2)C1]

A solution of tert-butyl 7-[(2-amino-5-bromopyridin-3-yl)oxy]-5-azaspiro[2.4]heptane-5-carboxylate (Preparation 36, 153 mg, 1.302 mmol) and zinc cyanide (153 mg, 1.302 mmol) in DMF (10 mL) was purged with nitrogen for 15 minutes followed by the addition of Pd₂(dba)₃ (143 mg, 0.156 mmol) and dppf (87 mg, 0.156 mmol). The reaction was heated to 100° C. for 16 hours before cooling and diluting with EtOAc. The solution was washed with water, brine, dried over sodium sulphate and concentrated in vacuo. The residue was purified using silica gel column chromatography eluting with 2-3% MeOH in DCM to afford the title compound as a yellow solid (130 mg, 30%).

MS m/z 331 [M+H]⁺

### Preparation 33

### (R)-tert-butyl 4-[(2-amino-5-cyanopyridin-3-yl)oxy]-3,3-dimethylpyrrolidine-1-carboxylate

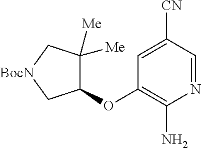 [SMILES: CC(C)(C)OC(=O)N1C[C@H](Oc2cc(C#N)cnc2N)C(C)(C)C1]

The title compound was prepared according to the method described for Preparation 32 using (R)-tert-butyl 4-[(2-amino-5-bromopyridin-3-yl)oxy]-3,3-dimethylpyrrolidine-1-carboxylate (Preparation 37).

¹H NMR (400 MHz, DMSO-d₆): δ ppm 1.02 (s, 3H), 1.11 (s, 3H), 1.39 (s, 9H), 3.11-3.20 (m, 2H), 3.40-3.45 (m, 1H), 3.68-3.76 (m, 1H), 4.50-4.51 (m, 1H), 6.80-7.00 (br s, 2H), 7.37 (s, 1H), 7.96 (s, 1H).

MS m/z 331 [M−H]⁻

### Preparation 34

### (S)-tert-butyl 4-[(2-amino-5-bromopyridin-3-yl)oxy]-3,3-difluoropyrrolidine-1-carboxylate

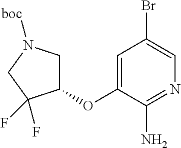

To a solution of (S)-tert-butyl 4-[(2-aminopyridin-3-yl)oxy]-3,3-difluoropyrrolidine-1-carboxylate (Preparation 40, 10.91 g, 35 mmol) in MeCN (100 mL) at 0-5° C. was added NBS (6.47 g, 36.3 mmol) and the reaction was stirred at this temperature for 30 minutes. The reaction was concentrated in vacuo and partitioned between EtOAc (200 mL) and water (200 mL). The organic layer was collected, washed with water (200 mL), brine (200 mL), dried over sodium sulphate and concentrated in vacuo. The residue was purified using silica gel column chromatography eluting with 20-30% EtOAc in heptanes followed by trituration with 10% TBME in heptanes to afford the title compound as a white solid (9.31 g, 68%).

¹H NMR (400 MHz, CDCl₃): δ ppm 1.50 (s, 9H), 3.60-4.00 (m, 4H), 4.60-4.78 (m, 1H), 4.80-5.00 (br s, 2H), 7.10 (s, 1H), 7.80 (s, 1H).

MS m/z 394 [M⁷⁹Br+H]⁺

### Preparation 35

### Racemic-tert-butyl 4-{[(2-amino-5-bromopyridin-3-yl)oxy]methyl}-3,3-difluoropyrrolidine-1-carboxylate

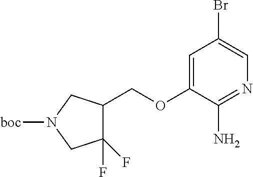

The title compound was prepared according to the method described for Preparation 34 using racemic-tert-butyl 4-{[(2-aminopyridin-3-yl)oxy]methyl}-3,3-difluoropyrrolidine-1-carboxylate (Preparation 41).

¹H NMR (400 MHz, DMSO-d₆): δ ppm 1.41 (s, 9H), 3.33-3.40 (m, 2H), 3.66-3.86 (m, 3H), 4.09-4.18 (m, 1H), 4.19-4.29 (m, 1H), 5.87 (s, 2H), 7.28 (d, 1H), 7.60 (d, 1H).

### Preparation 36

### tert-butyl 7-[(2-amino-5-bromopyridin-3-yl)oxy]-5-azaspiro[2.4]heptane-5-carboxylate

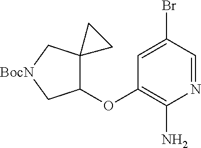 [SMILES: CC(C)(C)OC(=O)N1CC(Oc2cc(Br)cnc2N)C2(CC2)C1]

The title compound was prepared according to the method described for Preparation using tert-butyl-7-[(2-aminopyridin-3-yl)oxy]-5-azaspiro[2.4]heptane-5-carboxylate (Preparation 42).

¹H NMR (400 MHz, DMSO-d₆): δ ppm 0.45-0.54 (m, 1H), 0.61-0.64 (m, 1H), 0.73-0.76 (m, 1H), 0.82-0.84 (m, 1H), 1.38 (s, 9H), 3.02-3.04 (m, 1H), 3.20-3.23 (m, 1H), 3.37-3.48 (m, 2H), 3.49-3.81 (m, 1H), 4.88 (br s, 2H), 7.18 (d, 1H), 7.59 (d, 1H).

MS m/z 384 [M⁷⁹Br+H]⁺

### Preparation 37

### (R)-tert-butyl 4-[(2-amino-5-bromopyridin-3-yl)oxy]-3,3-dimethylpyrrolidine-1-carboxylate

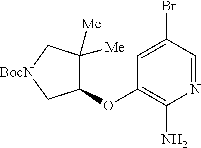 [SMILES: CC(C)(C)OC(=O)N1C[C@H](Oc2cc(Br)cnc2N)C(C)(C)C1]

The title compound was prepared according to the method described for Preparation 34 using (R)-tert-butyl 4-[(2-aminopyridin-3-yl)oxy]-3,3-dimethylpyrrolidine-1-carboxylate (Preparation 43).

MS m/z 388 [M⁸¹Br+H]⁺

### Preparation 38

### Racemic-tert-butyl 4-[(2-amino-5-bromopyridin-3-yl)oxy]-3-methoxy-3-methylpyrrolidine-1-carboxylate

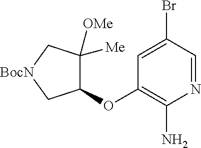 [SMILES: COC1(C)CN(C(=O)OC(C)(C)C)C[C@@H]1Oc1cc(Br)cnc1N]

The title compound was prepared according to the method described for Preparation 34 using racemic-tert-butyl-4-[(2-aminopyridin-3-yl)oxy]-3-methoxy-3-methylpyrrolidine-1-carboxylate (Preparation 44).

MS m/z 403 [M+H]⁺ and MS m/z 303 [M-Boc+H]⁺

### Preparation 39

### Racemic-1-{4-[(2-amino-5-bromopyridin-3-yl)methoxy]-3,3-difluoropyrrolidin-1-yl}-3,3-dimethylbutan-1-one

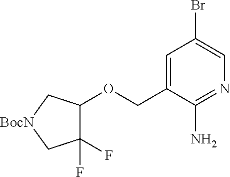 [SMILES: CC(C)(C)OC(=O)N1CC(OCc2cc(Br)cnc2N)C(F)(F)C1]

To a solution of 2-amino-5-bromo-3-pyridinemethanol (4 g, 19.7 mmol) in THF (25 mL) was added thionyl chloride (3 mL, 41 mmol) at 0° C. and the reaction was stirred warming to room temperature for 18 hours. The reaction was concentrated in vacuo to afford the intermediate chloride.

To a solution of NaH (2.2 g, 54 mmol) in THF (70 mL) was added racemic-tert-butyl-3-((tert-butyldimethylsilyl)oxy)-4-oxopyrrolidine-1-carboxylate (WO2014075392, 4.33 mL, 49 mmol) at 0° C. The reaction was stirred at 0° C. for 30 minutes. To the reaction was added the intermediate chloride (5.08 g, 19.7 mmol) and the reaction was heated to 60° C. for 4 hours. The reaction was cooled and partitioned between EtOAc and water. The organic layer was collected, dried over sodium sulphate and concentrated in vacuo. The residue was purified using silica gel column chromatography eluting with 50% EtOAc in heptanes to afford the title compound as a yellow oil that solidified on standing (700 mg, 85%).

¹H NMR (400 MHz, CDCl₃): δ ppm 1.47 (s, 9H), 3.54-3.56 (m, 1H), 3.63-3.83 (m, 3H), 4.01 (br s, 1H), 4.51-4.62 (m, 1H), 4.62-4.78 (m, 1H), 5.50 (br s, 2H), 7.53-7.56 (m, 1H), 8.08-8.10 (m, 1H).

### Preparation 40

### (S)-tert-butyl 4-[(2-aminopyridin-3-yl)oxy]-3,3-difluoropyrrolidine-1-carboxylate

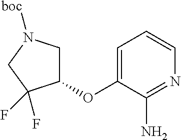

To a solution of (S)-tert-butyl 3,3-difluoro-4-[(2-nitropyridin-3-yl)oxy]pyrrolidine-1-carboxylate (Preparation 45, 14.28 g, 41.4 mmol) in IMS (140 mL) was added 10% Pd/C (1.4 g) and the reaction was hydrogenated at 50 psi at room temperature for 90 minutes. Further catalyst was added (1.4 g) and the reaction continued at room temperature at 50 psi for 18 hours. The reaction was filtered through celite and concentrated in vacuo to afford the title compound as an oil (11.91 g, 91%).

¹H NMR (400 MHz, CDCl₃): δ ppm 1.47 (s, 9H), 3.60-3.90 (m, 4H), 4.67-4.76 (m, 3H), 6.62-6.64 (m, 1H), 6.98-7.00 (m, 1H), 7.75-7.78 (m, 1H).

MS m/z 316 [M+H]⁺

### Preparation 41

### Racemic-tert-butyl 4-{[(2-aminopyridin-3-yl)oxy]methyl}-3,3-difluoropyrrolidine-1-carboxylate

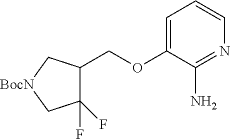 [SMILES: CC(C)(C)OC(=O)N1CC(COc2cccnc2N)C(F)(F)C1]

The title compound was prepared according to the method described for Preparation 40 using racemic-tert-butyl 3,3-difluoro-4-{[(2-nitropyridin-3-yl)oxy]methyl}pyrrolidine-1-carboxylate (Preparation 46) and taken on directly to the next step.

### Preparation 42

### tert-butyl 7-[(2-aminopyridin-3-yl)oxy]-5-azaspiro[2.4]heptane-5-carboxylate

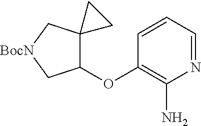 [SMILES: CC(C)(C)OC(=O)N1CC(Oc2cccnc2N)C2(CC2)C1]

The title compound was prepared according to the method described for Preparation using tert-butyl 7-[(2-nitropyridin-3-yl)oxy]-5-azaspiro[2.4]heptane-5-carboxylate (Preparation 47) in MeOH.

¹H NMR (400 MHz, DMSO-d₆): δ ppm 0.49-0.53 (m, 1H), 0.61-0.64 (m, 1H), 0.72-0.76 (m, 1H), 0.81-0.83 (m, 1H), 1.39 (s, 9H), 3.39-3.47 (m, 2H), 3.55-3.79 (m, 2H), 4.32-4.33 (m, 1H), 5.56-5.58 (br m, 2H), 6.44-6.47 (m, 1H), 6.95-6.97 (m, 1H), 7.51-7.52 (m, 1H).

MS m/z 306 [M+H]⁺

### Preparation 43

### (R)-tert-butyl 4-[(2-aminopyridin-3-yl)oxy]-3,3-dimethylpyrrolidine-1-carboxylate

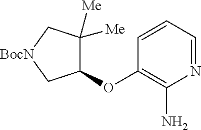 [SMILES: CC(C)(C)OC(=O)N1C[C@H](Oc2cccnc2N)C(C)(C)C1]

The title compound was prepared according to the method described for Preparation 40 using (R)-tert-butyl 3,3-dimethyl-4-[(2-nitropyridin-3-yl)oxy]pyrrolidine-1-carboxylate (Preparation 48) in MeOH.

MS m/z 306 [M−H]⁻

### Preparation 44

### Racemic-tert-butyl 4-[(2-aminopyridin-3-yl)oxy]-3-methoxy-3-methylpyrrolidine-1-carboxylate

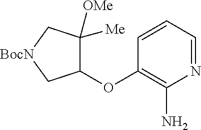 [SMILES: COC1(C)CN(C(=O)OC(C)(C)C)CC1Oc1cccnc1N]

The title compound was prepared according to the method described for Preparation 40 using racemic-tert-butyl-3-methoxy-3-methyl-4-[(2-nitropyridin-3-yl)oxy]pyrrolidine-1-carboxylate (Preparation 49) and taken directly on to the next step.

### Preparation 45

### (S)-tert-butyl 3,3-difluoro-4-[(2-nitropyridin-3-yl)oxy]pyrrolidine-1-carboxylate

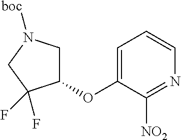

To a solution of (S)-tert-butyl 3,3-difluoro-4-hydroxypyrrolidine-1-carboxylate (Preparation 50, 10 g, 44.8 mmol) and 3-fluoro-2-nitropyridine (6.68 g, 47 mmol) in THF (200 mL) was added cesium carbonate (29.2 g, 89.6 mmol) and the reaction was heated to 65° C. for 18 hours. The reaction was cooled, diluted with water (400 mL) and extracted into EtOAc (2×400 mL). The combined organic extracts were washed with water (400 mL), brine (200 mL), dried over sodium sulphate and concentrated in vacuo. The residue was purified using silica gel column chromatography eluting with 20-50% EtOAc in heptanes to afford the title compound as a gum (14.28 g, 92%).

¹H NMR (400 MHz, CDCl₃): δ ppm 1.49 (s, 9H), 3.77-3.87 (m, 4H), 4.84 (br s, 1H), 7.55-7.65 (m, 2H), 8.22 (s, 1H).

### Preparation 46

### Racemic-tert-butyl 3,3-difluoro-4-{[(2-nitropyridin-3-yl)oxy]methyl}pyrrolidine-1-carboxylate

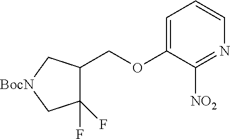 [SMILES: CC(C)(C)OC(=O)N1CC(COc2cccnc2[N+](=O)[O-])C(F)(F)C1]

The title compound was prepared according to the method described for Preparation using racemic-tert-butyl-3,3-difluoro-4-(hydroxymethyl)pyrrolidine-1-carboxylate (Preparation 51) and 3-fluoro-2-nitropyridine.

¹H NMR (400 MHz, CDCl₃): δ ppm 1.49 (s, 9H), 2.96-3.13 (m, 2H), 3.40-3.56 (m, 3H), 4.14-4.29 (m, 1H), 4.34-4.50 (m, 1H), 7.49-7.62 (m, 2H), 8.13-8.21 (m, 1H).

### Preparation 47

### tert-butyl 7-[(2-nitropyridin-3-yl)oxy]-5-azaspiro[2.4]heptane-5-carboxylate

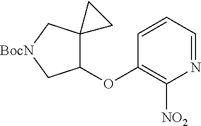 [SMILES: CC(C)(C)OC(=O)N1CC(Oc2cccnc2[N+](=O)[O-])C2(CC2)C1]

The title compound was prepared according to the method described for Preparation 45 using tert-butyl 7-hydroxy-5-azaspiro[2.4]heptane-5-carboxylate (Preparation 55) and 3-fluoro-2-nitropyridine.

¹H NMR (400 MHz, DMSO-d₆): δ ppm 0.68-0.79 (m, 4H), 1.39 (s, 9H), 2.95-3.05 (m, 1H), 3.39-3.72 (m, 4H), 7.72-7.75 (m, 1H), 7.96-7.98 (m, 1H), 8.12-8.13 (m, 1H).

### Preparation 48

### (R)-tert-butyl 3,3-dimethyl-4-[(2-nitropyridin-3-yl)oxy]pyrrolidine-1-carboxylate

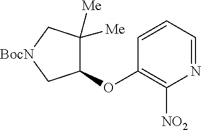 [SMILES: CC(C)(C)OC(=O)N1C[C@H](Oc2cccnc2[N+](=O)[O-])C(C)(C)C1]

The title compound was prepared according to the method described for Preparation 45 using tert-butyl-(R)-4-hydroxy-3,3-dimethylpyrrolidine-1-carboxylate (Preparation 54) and 3-fluoro-2-nitropyridine.

¹H NMR (400 MHz, DMSO-d₆): δ ppm 1.04 (s, 3H), 1.13 (s, 3H), 1.39 (s, 9H), 2.97-3.22 (m, 2H), 3.32-3.39 (m, 1H), 3.63-3.72 (m, 1H), 4.40-4.41 (m, 1H), 6.45-6.48 (m, 1H), 6.98-7.00 (m, 1H), 7.50-7.51 (m, 1H).

### Preparation 49

### Racemic-tert-butyl 3-methoxy-3-methyl-4-[(2-nitropyridin-3-yl)oxy]pyrrolidine-1-carboxylate

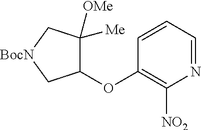 [SMILES: COC1(C)CN(C(=O)OC(C)(C)C)CC1Oc1cccnc1[N+](=O)[O-]]

The title compound was prepared according to the method described for Preparation using racemic-tert-butyl 4-hydroxy-3-methoxy-3-methyl pyrrolidine-1-carboxylate (Preparation 59) and 3-fluoro-2-nitropyridine.

¹H NMR (400 MHz, CDCl₃): δ ppm 1.30-1.70 (m, 12H), 3.30 (s, 3H), 3.30-3.85 (m, 4H), 4.60-4.65 (m, 1H), 7.40-7.50 (m, 2H), 8.15 (s, 1H).

### Preparation 50

### (S)-tert-butyl 3,3-difluoro-4-hydroxypyrrolidine-1-carboxylate

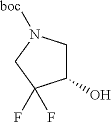

To a solution of (S)-tert-butyl 3-((tert-butyldimethylsilyl)oxy)-4-oxopyrrolidine-1-carboxylate (WO2010111057, 60 g, 0.19 mol) in DCM (300 mL) was added morpholinosulphur trifluoride (47 mL, 0.386 mol) under nitrogen. The reaction was stirred at room temperature for 18 hours before pouring into ice in a solution of sodium bicarbonate (250 g) in water (1 L). Upon cessation of the gas evolution, the organic layer was collected, washed with brine (100 mL), dried over sodium sulphate and concentrated in vacuo. The residue was purified using silica gel column chromatography eluting with 10% EtOAc in heptanes. To a solution of the residue (98 g, 0.291 mol) in THF (350 mL) was added a 1M solution of TBAF in THF (350 mL, 0.350 mol) and the reaction was stirred at room temperature for 1 hour. The reaction was diluted with water (3.9 L) and extracted into EtOAc (2.9 L). The organic layer was collected, washed with water (3.9 L), dried over sodium sulphate and concentrated in vacuo. The residue was purified using silica gel column chromatography eluting with 20% EtOAc in heptanes to afford the title compound as a gum that solidified on standing (57 g, 88%).

¹H NMR (400 MHz, CDCl₃): δ ppm 1.46 (s, 9H), 3.44-3.53 (m, 1H), 3.66-3.77 (m, 3H), 4.21-4.27 (m, 1H).

### Preparation 51

### Racemic-tert-butyl 3,3-difluoro-4-(hydroxymethyl)pyrrolidine-1-carboxylate

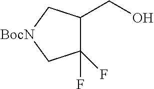 [SMILES: CC(C)(C)OC(=O)N1CC(CO)C(F)(F)C1]

To an ice-cooled solution of racemic-1-tert-butyl 3-ethyl 4,4-difluoropyrrolidine-1,3-dicarboxylate (Preparation 52, 918 mg, 3.29 mmol) in THF (11 mL) was added a 1M solution of LiAlH₄ in THF (3.62 mL, 3.62 mmol) and the reaction was stirred for 5 minutes. A solution of Rochelle's salt (10 mL) was added and the reaction extracted into EtOAc. The organic layer was separated, dried over magnesium sulphate and concentrated in vacuo. The residue was taken on directly to the next step as the title compound (749 mg, 96%).

### Preparation 52

### Racemic-1-tert-butyl 3-ethyl 4,4-difluoropyrrolidine-1,3-dicarboxylate

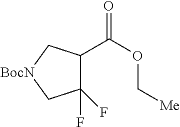 [SMILES: CCOC(=O)C1CN(C(=O)OC(C)(C)C)CC1(F)F]

To a solution of racemic-ethyl 1-benzyl-4,4-difluoropyrrolidine-3-carboxylate (Preparation 53, 885 mg, 3.29 mmol) in EtOH (20 mL) was added 20% palladium hydroxide on carbon (85 mg, 0.68 mmol) and the reaction was purged with hydrogen three times. Di-tert-butyldicarboxylate (860 mg, 3.94 mmol) was added and the reaction stirred at room temperature under a balloon of hydrogen for 18 hours. The reaction was filtered through Celite and concentrated in vacuo to afford the title compound as a colourless oil (918 mg, 100%).

¹H NMR (400 MHz, CDCl₃): δ ppm 1.20 (t, 3H), 1.45 (s, 9H), 3.25-3.40 (m, 1H), 3.60-3.80 (br m, 4H), 4.10-4.20 (m, 2H).

### Preparation 53

### Racemic-ethyl 1-benzyl-4,4-difluoropyrrolidine-3-carboxylate

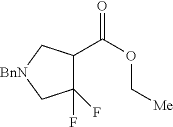 [SMILES: CCOC(=O)C1CN(Cc2ccccc2)CC1(F)F]

To a solution of ethyl-3,3,3-trifluoropropionate (2.50 g, 16 mmol) in deuterochloroform (15 mL) was added triethylamine (3.35 mL, 24 mmol) followed by trimethylsiyl triflate (4.45 mL, 24 mmol) dropwise and the reaction was stirred at room temperature for 1 hour. The reaction was cooled to 0° C. and treated with zirconium tetrachloride (565 mg, 2.4 mmol) followed by stirring at room temperature for 1.5 hours. The reaction was cooled to 0° C. and quenched by the addition of water (20 mL). The organic layer was collected, dried over magnesium sulphate and the filtrate cooled to 0° C. To the solution was added N-benzyl-N-(methoxymethyl)-N-(trimethylsilylmethyl)amine (2.50 mL, 9.4 mmol) followed by TFA (0.1 mL) and the reaction was stirred warming to room temperature for 18 hours. The reaction was quenched by the addition of saturated aqueous NaHCO₃ solution, the organic layer was separated, dried over magnesium sulphate and concentrated in vacuo. The reaction was purified using silica gel column chromatography eluting with 0-40% EtOAc in heptanes to afford the title compound (468 mg, 27%).

¹H NMR (400 MHz, CDCl₃): δ ppm 1.20-1.22 (m, 3H), 2.60-2.70 (m, 1H), 2.80-2.90 (m, 1H), 3.00-3.10 (m, 2H), 3.10-3.20 (m, 1H), 3.25-3.35 (m, 1H), 3.60-3.70 (m, 2H), 4.05-4.20 (m, 2H), 7.15-7.30 (m, 5H).

### Preparation 54

### tert-butyl-(R)-4-hydroxy-3,3-dimethylpyrrolidine-1-carboxylate

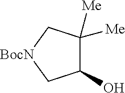 [SMILES: CC(C)(C)OC(=O)N1C[C@H](O)C(C)(C)C1]

To a solution of (R)-4,4-dimethylpyrrolidin-3-ol (WO2009061879, 1.49 g, 12.96 mmol) in THF (20 mL) was added triethylamine (4.51 mL, 32.29 mmol) followed by di-tert-butyldicarbonate (4.15 mL, 19.44 mmol) and the reaction was stirred at room temperature for 2 hours. The reaction was concentrated in vacuo and partitioned between EtOAc and water. The organic layer was separated, washed with water, brine, dried over sodium sulphate and concentrated in vacuo. The residue was purified using silica gel column chromatography eluting with 6-7% MeOH in DCM to afford the title compound as a yellow oil (2 g, 72%).

¹H NMR (400 MHz, CDCl₃): δ ppm 0.99 (s, 3H), 1.05 (s, 3H), 1.44 (s, 9H), 3.07-3.31 (m, 3H), 3.59-3.68 (m, 1H), 3.78-3.80 (m, 1H).

### Preparation 55

### tert-butyl 7-hydroxy-5-azaspiro[2.4]heptane-5-carboxylate

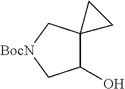 [SMILES: CC(C)(C)OC(=O)N1CC(O)C2(CC2)C1]

The title compound was prepared according to the method described for Preparation 54 using racemic-5-azaspiro[2.4]heptan-7-ol (US20100239576) and taken on directly to the next step.

### Preparation 56

### (3S,4S)-tert-butyl 3-methyl-4-[(methylsulfonyl)oxy]pyrrolidine-1-carboxylate

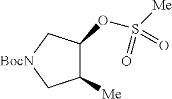 [SMILES: C[C@H]1CN(C(=O)OC(C)(C)C)C[C@H]1OS(C)(=O)=O]

To a solution of tert-butyl 3-hydroxy-4-methylpyrrolidine-1-carboxylate (WO2009013211, 530 mg, 2.64 mmol) in DCM (15 mL) was added triethylamine (0.55 mL, 3.95 mmol) followed by mesyl chloride (0.245 mL, 3.16 mmol) at −20° C. The reaction was stirred at −20° C. for 3 hours before being filtered through Celite and concentrating in vacuo. The residue was washed with hexanes to afford the title compound as a brown solid (1 g, 96%) that was taken on directly to the next step.

### Preparation 57

### Methyl 6-amino-5-(benzyloxy)pyridine-3-carboxylate

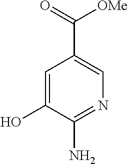 [SMILES: COC(=O)c1cnc(N)c(O)c1]

A solution of methyl 6-amino-5-(benzyloxy)pyridine-3-carboxylate (Preparation 58, 1.6 g, 6.19 mmol) in MeOH (50 mL) was degassed with argon before the addition of 20% Pd/C (300 mg). The reaction was stirred at room temperature under 20 psi of hydrogen for 3 hours. The reaction was filtered through Celite, washed with 20% MeOH in DCM and concentrated in vacuo. The residue was washed with hexanes to afford the title compound as a brown solid (1 g, 96%).

¹H NMR (400 MHz, DMSO-d₆): δ ppm 3.73 (s, 3H), 6.40 (s, 2H), 7.23 (s, 1H), 8.08 (s, 1H), 9.92 (s, 1H).

### Preparation 58

### Methyl 6-amino-5-(benzyloxy)pyridine-3-carboxylate

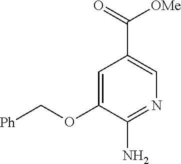 [SMILES: COC(=O)c1cnc(N)c(OCc2ccccc2)c1]

The title compound was prepared according to the method described for Preparation 26 using 3-(benzyloxy)-5-bromopyridin-2-amine in DMA:MeOH 1:1 under 115 psi carbon monoxide at 110° C. The residue was purified using silica gel column chromatography eluting with 30% EtOAc in heptanes.

¹H NMR (400 MHz, DMSO-d₆): δ ppm 3.76 (s, 3H), 5.20 (s, 2H), 6.68 (br s, 2H), 7.31-7.52 (m, 6H), 8.19 (s, 1H).

MS m/z 259 [M+H]⁺

### Preparation 59

### Racemic-tert-butyl 4-hydroxy-3-methoxy-3-methylpyrrolidine-1-carboxylate

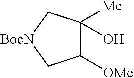 [SMILES: COC1CN(C(=O)OC(C)(C)C)CC1(C)O]

To a solution of racemic-tert-butyl 1-methyl-6-oxa-3-azabicyclo[3.1.0]hexane-3-carboxylate (Preparation 60, 1.07 mg, 3.82 mmol) in MeOH (15 mL) was added PTSA (73 mg, 0.382 mmol) and the reaction was heated to reflux for 2 hours. The reaction was cooled and concentrated in vacuo. The residue was purified using silica gel column chromatography eluting with 0-50% EtOAc in heptanes to afford the title compound (980 mg, >100%) that was taken on directly to the next step.

### Preparation 60

### Racemic-tert-butyl 1-methyl-6-oxa-3-azabicyclo[3.1.0]hexane-3-carboxylate

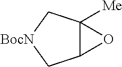 [SMILES: CC(C)(C)OC(=O)N1CC2OC2(C)C1]

To a solution of tert-butyl 3-methyl-2,5-dihydro-1H-pyrrole-1-carboxylate (Org. Lett. (2010), 12 (5), 984-987, 1.34 g, 7.33 mmol) in dioxane (37 mL) and water (9 mL) was added NBS (1.57 g, 8.8 mmol) and the reaction was stirred at room temperature for 1.5 hours. NaOH (352 mg, 8.8 mmol) was added and the reaction was sonicated and stirred for 30 minutes. The reaction was diluted with water and extracted into EtOAc. The organic layer was collected, dried over sodium sulphate and concentrated in vacuo. The residue was purified using silica gel column chromatography eluting with 0-25% EtOAc in heptanes to afford the title compound as an oil (913 mg, 63%) that was taken on directly to the next step.

### Biological Activity

Isolated TRK Enzyme assays use the HTRF KinEASE-TK kit (Cisbio Cat#62TKOPEJ) with recombinant His-tagged cytoplasmic domains of TRKA receptor sourced from Invitrogen (see table below). This activity-assay measures the phosphorylation of tyrosine residues within a substrate from the HTRF kit which has been validated by Cisbio for a variety of tyrosine kinases including the TRK receptors.

Assay details:

| | Invitrogen | Amino | FAC | FAC | Assay Reaction |
|---|---|---|---|---|---|
| Target | Cat# | acids | enzyme | ATP | Time |
| TRKA | PV3144 | aa 441- | 4 nM | 40 uM | 35 min |
| | (NTRK1) | 796 | | | |

0.5 mM stock solutions of test compounds are prepared and serially diluted in 100% DMSO. A standard curve using the compound of Example 135 disclosed in WO2005/116035 of 150 uM is also prepared on each test plate. High percentage effect (HPE) is defined by 150 uM (using the compound of Example 135 as disclosed in WO2005/116035) and 0% effect (ZPE) is defined by 100% DMSO. Greiner low volume black plates containing 0.2 ul of serially diluted compound, standard and HPE/ZPE are created using the Bravo nanolitre dispenser. 1× enzyme buffer is prepared from 5× Enzymatic Buffer from the Cisbio KinEASE TK kit using MilliQ water. The buffer is then supplemented with 10 mM MgCl and 2 mM DTT (both from Sigma). In the case of TRKB, the buffer is also supplemented with 125 nM Supplement Enzymatic Buffer (SEB) from the Cisbio kit. 2×FAC of enzyme and 2×FAC ATP diluted in 1× complete enzyme buffer is incubated at room temperature for 20 minutes to preactivate the enzyme. Following this preactivation step, 5 ul/well of enzyme+ATP mix is added using a Multidrop Micro to the assay plate, spotted with 0.2 ul 100% DMSO compound. This is left for 20 mins at room temperature before adding 5 ul of 2 uM TK-substrate-Biotin (from the Cisbio kit) diluted in 1× enzyme buffer (1 uM FAC) using the Multidrop Micro. The reaction is incubated at room temperature for the optimized assay reaction time (see table). The reaction is stopped by adding 10 ul/well HTRF Detection Buffer containing 0.25 uM Streptavidin-XL665 (0.125 uM FAC) and 1:200 TK Antibody-Cryptate using a Multidrop.

After the Detection Reagent addition, plates are covered and incubated at room temperature for 60 minutes. HTRF signal is read using an Envision reader, measured as a ratio of emissions at two different wavelengths, 620 nm and 665 nm. Any compound that inhibits the action of the TRK kinase will have a lower fluorescence ratio value 665/620 nM than compounds which do not inhibit the TRK kinase. Test compound data are expressed as percentage inhibition defined by HPE and ZPE values for each plate.

Percentage inhibition in the presence of test compound is plotted against compound concentration on a log scale to determine an IC₅₀ from the resultant sigmoid curve. Cell Based Assays were carried out using Cell lines from DiscoveRx utilising their PathHunter technology and reagents in an antagonist assay:

| Target | DiscoveRx cell line Cat# | Cognate Neurotrophin |
|---|---|---|
| TRKA | 93-0462C3 | NGF |
| TRKA co expressed | 93-0529C3 | NGF |
| with p75 | | |

The assays are based upon DiscoveRx's proprietary Enzyme Fragment Complementation (EFC) technology. In the case of the TRK cell lines, the enzyme acceptor (EA) protein is fused to a SH2 protein and the TRK receptor of interest has been tagged with a Prolink tag.

Upon neurotrophin binding, the TRKA receptor becomes phosphorylated, and the tagged SH2 protein binds. This results in functional complementation and restored β-Galactosidase activity which is can be measured using the luminescent Galacton Star substrate within the PathHunter reagent kits.

Generally, small molecule inhibitors bind to the kinase domain so are not competing with the neurotrophin (agonist) which binds to an extracellular site. This means that the IC₅₀ is a good measure of affinity and should be unaffected by concentration neurotrophin stimulant.

Cryopreserved PathHunter cells are used from either in-house produced batches or bulk batches bought directly from DiscoveRx. Cryopreserved cells are resuscitated, spun 1000 rpm for 4 min to remove freezing media, and resuspended in MEM+0.5% horse serum (both Invitrogen) to 5e⁵cells/ml. The cells are then plated using a Multidrop into Greiner white tissue culture treated plates at 20 ul/well and incubated for 24 h at 37° C., 5% CO₂, high humidity. On the day of the assay, the cell plates are allowed to cool to room temperature for 30 min prior to the assay.

4 mM stock solutions of test compounds are prepared and serially diluted in 100% DMSO. A standard curve using the compound of Example 135, WO2005/116035 at a top concentration of 150 uM is also prepared on each test plate. High percentage effect (HPE) is defined by 150 uM of the compound of Example 135, WO2005/116035 and 0% effect (ZPE) is defined by 100% DMSO. Plates containing 1 ul of serially diluted compound, standard and HPE/ZPE are diluted 1/66 in assay buffer (PBS minus Ca²⁺, minus Mg²⁺ with 0.05% pluronic F127) using a Wellmate. Using a Platemate Plus, 5 ul of 1/66 diluted test compounds is then transferred to the cell plate and allowed to reach equilibrium by incubating for 30 min at room temperature before addition of agonist stimulus: 10 ul/well of 2 nM (0.571 nM FAC) of the cognate neurotrophin (Peprotech) diluted in agonist buffer (HBSS with 0.25% BSA). Final assay concentration of the test compounds is 8.66 μM, (the compound of Example 135, WO2005/116035 FAC is 0.325 uM). The plates are left at room temperature for a further 2 hours before addition of 10 ul of the DiscoveRx PathHunter detection reagent (made up by adding 1 part Galacton Star, 5 parts Emerald II and 19 parts Cell Assay Buffer as per the manufacturer's instructions).

After reagent addition, plates are covered and incubated at room temperature for 60 minutes. Luminescence signal is read using an Envision. Test compound data are expressed as percentage inhibition defined by HPE and ZPE values for each plate. Percentage inhibition in the presence of test compound is plotted against compound concentration on a log scale to determine an IC₅₀ from the resultant sigmoid curve.

Below are TrkA IC₅₀ data generated using the PV3144 TrkA enzyme assay. Where more than one reading was taken, the arithmetic mean is presented.

| | TRKA |
|---|---|
| Ex | IC₅₀ (nM) |
| 1 | 7.9 |
| 2 | 10 |
| 3 | 10.1 |
| 4 | |
| 5 | 10.8 |
| 6 | 630 |
| 7 | |
| 8 | 148 |
| 9 | |
| 10 | 8660 |
| 11 | 11.6 |
| 12 | 1250 |
| 13 | 30.3 |
| 14 | |
| 15 | 15.8 |
| 16 | 72.4 |
| 17 | 105 |
| 18 | 110 |
| 19 | 11.8 |
| 20 | 12.3 |
| 21 | 126 |
| 22 | 330 |
| 23 | 2100 |
| 24 | 55.5 |
| 25 | 129 |

All publications cited in this application are each herein incorporated by reference in their entirety.

Although the invention has been described above with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

1. A compound of Formula I: 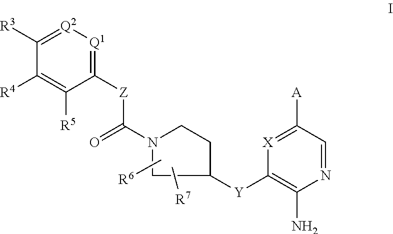
or a pharmaceutically acceptable salt thereof, wherein
Q¹ is N or CR¹,
Q² is N or CR²,
R¹, R², R⁴ and R⁵ are each independently H, halogen, CN, OH, NH₂, C₁₋₃ alkyl optionally substituted by one or more F, C₃₋₇ cycloalkyloxy optionally substituted by one or more F, or C₁₋₃ alkoxy optionally substituted by one or more F,
R³ is H, halogen, CN, C₁₋₄ alkyl optionally substituted by one or more F, C₁₋₄ alkoxy optionally substituted by one or more F, C₃₋₇ cycloalkyloxy optionally substituted by one or more F, or C₁₋₄ alkylthio optionally substituted by one or more F,
With the proviso that at least 2 of R¹, R², R³, R⁴ and R⁵ are H,
Y is O, CH₂O or OCH₂
R⁶ and R⁷ can be attached at any point on the ring and are independently H, F, CN, OH, NH₂, C₁₋₃ alkyl optionally substituted by one or more F, or C₁₋₃ alkoxy optionally substituted by one or more F,
or R⁶ and R⁷ can be taken together, with the atoms to which they are attached, to form a 3- to 7-membered cycloalkane ring,
X is CR¹⁰¹ or N,
R¹⁰¹ is H or C₁₋₃ alkyl,
Z is CH₂, CH(CH₃), NH or O,
A is C(O)NR¹⁰³R¹⁰⁴,
R¹⁰³ and R¹⁰⁴ are each independently selected from H, (C₁₋₆ alkyl optionally substituted by OH, C₁₋₆ alkoxy, CN or by one or more F), and (C₃₋₇ cycloalkyl optionally substituted by OH, C₁₋₆ alkoxy or by one or more F).

2. The compound according to claim 1, or a pharmaceutically acceptable salt thereof, wherein Q¹ is CH.

3. The compound according to claim 1, or a pharmaceutically acceptable salt thereof, wherein Q² is CH.

4. The compound according to claim 1, or a pharmaceutically acceptable salt thereof, wherein R⁴ is H.

5. The compound according to claim 1, or a pharmaceutically acceptable salt thereof, wherein R⁵ is H.

6. The compound according to claim 1, or a pharmaceutically acceptable salt thereof, wherein R³ is halogen, C₁₋₄ alkyl optionally substituted by one or more F, C₁₋₄ alkoxy optionally substituted by one or more F, or C₃₋₇ cycloalkyloxy optionally substituted by one or more F, or C₁₋₄ alkylthio optionally substituted by one or more F.

7. The compound according to claim 6, or a pharmaceutically acceptable salt thereof, wherein R³ is C₁₋₄ alkoxy optionally substituted by one or more F.

8. The compound according to claim 7, or a pharmaceutically acceptable salt thereof, wherein R³ is OCF₃.

9. The compound according to claim 1, or a pharmaceutically acceptable salt thereof, wherein R⁶ and R⁷ can be attached at any point on the ring and are independently H, F, methyl optionally substituted by one or more F, ethyl optionally substituted by one or more F, or methoxy optionally substituted by one or more F,
or R⁶ and R⁷ can be taken together, with the atoms to which they are attached, to form a cyclopropyl ring.

10. The compound according to claim 9, or a pharmaceutically acceptable salt thereof, wherein R⁶ is H, F or methyl and R⁷ is F, methyl or methoxy, or R⁶ and R⁷ together are cyclopropyl.

11. The compound according to claim 1, or a pharmaceutically acceptable salt thereof, wherein X is CR¹⁰¹.

12. The compound or according to claim 11, or a pharmaceutically acceptable salt thereof, wherein X is CH.

13. The compound according to claim 1, or a pharmaceutically acceptable salt thereof, wherein Y is O.

14. The compound according to claim 1, or a pharmaceutically acceptable salt thereof, wherein R¹⁰³ and R¹⁰⁴ are each independently selected from H and C₁₋₆ alkyl optionally substituted by OH or CN.

15. The compound according to claim 14, or a pharmaceutically acceptable salt thereof, wherein R¹⁰³ is H, methyl or ethyl.

16. The compound according to claim 14, or a pharmaceutically acceptable salt thereof, wherein R¹⁰⁴ is selected from H, methyl, ethyl, 2-hydroxyethyl, 2,2-dimethyl-2-hydroxyethyl or cyanomethyl.

17. The compound according to claim 1, of formula IA: 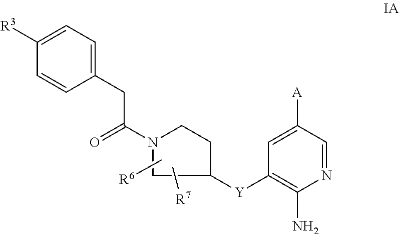
or a pharmaceutically acceptable salt thereof.

18. The compound according to claim 1, of formula IB 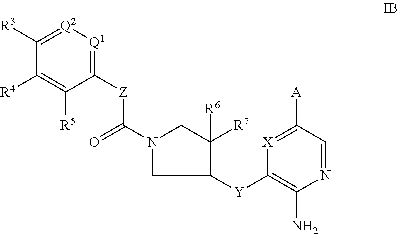
or a pharmaceutically acceptable salt thereof.

19. A compound selected from:
(S)-6-amino-5-[(4,4-difluoro-1-{[4-(trifluoromethoxy)phenyl]acetyl}pyrrolidin-3-yl)oxy]-N-methylpyridine-3-carboxamide;
(R)-6-amino-5-[(4,4-difluoro-1-{[4-(trifluoromethoxy)phenyl]acetyl}pyrrolidin-3-yl)oxy]-N-methylpyridine-3-carboxamide;
(R)-6-amino-5-{[(4,4-difluoro-1-{[4-(trifluoromethoxy)phenyl]acetyl}pyrrolidin-3-yl)oxy]methyl}-N-methylpyridine-3-carboxamide;
(S)-6-amino-5-{[(4,4-difluoro-1-{[4-(trifluoromethoxy)phenyl]acetyl}pyrrolidin-3-yl)oxy]methyl}-N-methylpyridine-3-carboxamide;
(R)-6-amino-5-{[(4,4-difluoro-1-{[4-(trifluoromethoxy)phenyl]acetyl}pyrrolidin-3-yl)oxy]methyl}pyridine-3-carboxamide;
(S)-6-amino-5-{[(4,4-difluoro-1-{[4-(trifluoromethoxy)phenyl]acetyl}pyrrolidin-3-yl)oxy]methyl}pyridine-3-carboxamide;
(R)-6-amino-5-[(4,4-difluoro-1-{[4-(trifluoromethoxy)phenyl]acetyl}pyrrolidin-3-yl)oxy]-N-(2-hydroxyethyl)pyridine-3-carboxamide;
(S)-6-amino-5-[(4,4-difluoro-1-{[4-(trifluoromethoxy)phenyl]acetyl}pyrrolidin-3-yl)oxy]-N-(2-hydroxyethyl)pyridine-3-carboxamide;
(R)-6-amino-5-[(4,4-difluoro-1-{[4-(trifluoromethoxy)phenyl]acetyl}pyrrolidin-3-yl)oxy]-N-(2-hydroxy-2-methylpropyl)pyridine-3-carboxamide;
(S)-6-amino-5-[(4,4-difluoro-1-{[4-(trifluoromethoxy)phenyl]acetyl}pyrrolidin-3-yl)oxy]-N-(2-hydroxy-2-methylpropyl)pyridine-3-carboxamide;
(R)-6-amino-5-[(4,4-difluoro-1-{[4-(trifluoromethoxy)phenyl]acetyl}pyrrolidin-3-yl)methoxy]-N-methylpyridine-3-carboxamide;
(S)-6-amino-5-[(4,4-difluoro-1-{[4-(trifluoromethoxy)phenyl]acetyl}pyrrolidin-3-yl)methoxy]-N-methylpyridine-3-carboxamide;
6-amino-5-[(4,4-difluoro-1-{[4-(trifluoromethoxy)phenyl]acetyl}pyrrolidin-3-yl)methoxy]pyridine-3-carboxamide;
(S)-6-amino-5-[(4,4-difluoro-1-{[4-(trifluoromethoxy)phenyl]acetyl}pyrrolidin-3-yl)methoxy]pyridine-3-carboxamide;
(R)-6-amino-5-[(4,4-difluoro-1-{[4-(trifluoromethoxy)phenyl]acetyl}pyrrolidin-3-yl)methoxy]pyridine-3-carboxamide;
6-amino-N-methyl-5-[(5-{[4-(trifluoromethoxy)phenyl]acetyl}-5-azaspiro[2.4]hept-7-yl)oxy]pyridine-3-carboxamide;
(3R,4S)-6-amino-N-methyl-5-[(4-methyl-1-{[4-(trifluoromethoxy)phenyl]acetyl}pyrrolidin-3-yl)oxy]pyridine-3-carboxamide;
6-amino-5-[(5-{[4-(trifluoromethoxy)phenyl]acetyl}-5-azaspiro[2.4]hept-7-yl)oxy]pyridine-3-carboxamide;
(R)-6-amino-5-[(4,4-dimethyl-1-{[4-(trifluoromethoxy)phenyl]acetyl}pyrrolidin-3-yl)oxy]pyridine-3-carboxamide;
(R)-6-amino-5-[(4,4-dimethyl-1-{[4-(trifluoromethoxy)phenyl]acetyl}pyrrolidin-3-yl)oxy]-N-methylpyridine-3-carboxamide;
(3R,4S)-6-amino-N-ethyl-5-[(4-methyl-1-{[4-(trifluoromethoxy)phenyl]acetyl}pyrrolidin-3-yl)oxy]pyridine-3-carboxamide;
(3R,4S)-6-amino-N-(cyanomethyl)-5-[(4-methyl-1-{[4-(trifluoromethoxy)phenyl]acetyl}pyrrolidin-3-yl)oxy]pyridine-3-carboxamide;
(3R,4S)-6-amino-N-(2-hydroxy-2-methylpropyl)-5-[(4-methyl-1-{[4-(trifluoromethoxy)phenyl]acetyl}pyrrolidin-3-yl)oxy]pyridine-3-carboxamide;
6-amino-5-[(4-methoxy-4-methyl-1-{[4-(trifluoromethoxy)phenyl]acetyl}pyrrolidin-3-yl)oxy]-N-methylpyridine-3-carboxamide;
6-amino-N,N-diethyl-5-[(4-methoxy-4-methyl-1-{[4-(trifluoromethoxy)phenyl]acetyl}pyrrolidin-3-yl)oxy]pyridine-3-carboxamide;
or a pharmaceutically acceptable salt thereof.

20. A pharmaceutical composition comprising a compound of formula (i), or a pharmaceutically acceptable salt thereof, as defined in claim 1, and a pharmaceutically acceptable carrier.

21. A method of treating a disease or condition for which a Trk receptor antagonist is indicated, comprising administering to a mammal in need thereof a compound of formula (i), or a pharmaceutically acceptable salt thereof, as defined in claim 1.

22. A method of treatmenting pain or cancer in a mammal, comprising treating said mammal with an effective amount of a compound of formula (I) or a pharmaceutically acceptable salt thereof, as defined in claim 1.

23. The method according to claim 21, wherein said compound of formula (i) or pharmaceutically acceptable salt thereof is administered in combination with a further drug substance.
